(19) United States
(12) Patent Application Publication
(10) Pub. No.: US 2002/0095732 A1
(43) Pub. Date: Jul. 25, 2002

(54) Compositions for oxidation dyeing keratinous fibers comprising at least one oxidation precursor, and at least one direct dye, and dyeing methods

(76) Inventors: Sylvain Kravtchenko, Asnieres (FR); Alain Lagrange, Coupvray (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/836,600

(22) Filed: Apr. 18, 2001

(30) Foreign Application Priority Data
Apr. 18, 2000 (FR) 00 04991

(51) Int. Cl. A61K 7/13

(52) U.S. Cl. 8/405; 8/406; 8/421; 8/423

(57) ABSTRACT

Compositions for oxidation dyeing keratinous fibers, for example, human keratinous fibers such as hair, comprising, in a medium suitable for dyeing, at least one oxidation precursor chosen from 1-(4-aminophenyl)pyrrolidines and acid addition salts of formula (I), and at least one direct dye and processes comprising such compositions.

### DESCRIPTION OF THE INVENTION

The present invention relates to compositions for oxidation dyeing keratinous fibers, such as, human keratinous fibers, for example, human hair comprising, in a medium suitable for dyeing, at least one oxidation dye precursor chosen from 1-(4-aminophenyl)pyrrolidines and acid addition salts thereof, and at least one direct dye chosen from nitrobenzene dyes and cationic dyes comprising a quaternized nitrogen atom and a —Z═D— bond, wherein Z and D, which are identical or different, are each chosen from a nitrogen atom and a —CH— group, and dyeing methods for such compositions.

It is known to dye keratinous fibers, such as human hair, with dyeing compositions comprising oxidation dye precursors, such as, ortho-phenylenediamines, para-phenylenediamines, ortho-a aminophenols, para-aminophenols and heterocyclic bases, generally called oxidation bases. The oxidation dye precursors, or oxidation bases, may be colorless or weakly colored compounds. When these compounds are combined with oxidizing product, dye and colored compounds may form by a process of oxidative condensation.

It is also known that it is possible to vary the shades obtained with these oxidation bases by combining them with couplers or color modifiers, the latter chosen, for example, from aromatic meta-diamines, meta-aminophenols, meta-diphenols and certain heterocyclic compounds.

The variety of molecules used in oxidation bases and couplers can contribute to a palette very rich in color.

The “permanent” colors obtained by using these oxidation dyes may moreover satisfy at least one of a number of objectives. Thus, it should, for example, satisfy at least one of the following: be without toxicological drawbacks, make it possible to obtain shades in the desired intensity and exhibit good resistance towards external agents (at least one of: light, adverse weather conditions, washing, permanent waving, perspiration, and rubbing).

The dyes may also cover white and grey hair and may also be the least selective possible, that is to say, make it possible to obtain the smallest possible differences in color right along the same keratinous fiber, which may indeed be differently sensitised (i.e. damaged) between its tip and its root.

Thus, the permanent dyeing of hair with at least one para-phenylenediamine (PPD) coupling product in the presence of hydrogen peroxide is known and is in very widespread use. Nevertheless, better tolerated oxidation bases have been sought and proposed as alternatives to PPD. Among them, the tertiary base N,N-bis(β-hydroxyethyl)-para-phenylenediamine has been used in commercial hair-dyeing products.

Nevertheless, the colors obtained using these compositions may not always be entirely satisfactory. For example, the processes may affect the intensity (strength) of the colors, chromatic or the color resistance to the various attacking factors to which the hair may be subjected.

However, the inventors have discovered that combination of at least one oxidation base(oxidation dye precursor) chosen from 1-(4-aminophenyl)pyrrolidines, acid addition salts thereof and at least one (as used herein, “at least one” means one or more and thus includes mixtures and combinations) direct dye make it possible to obtain oxidation dyes leading to at least one of the following advantages: intense colors without giving rise to any significant degradation of the keratinous fibers, colors which are not very selective, colors which are chromatic and aesthetically pleasing and colors which are resistant to the various treatments to which keratinous fibers, such as hair, may be subjected.

As used herein, the term “lower alkyl” means an alkyl chosen from saturated and unsaturated, branched and unbranched (C₁-C₆)alkyl groups.

One embodiment of the invention is a composition for oxidation dyeing keratinous fibers, for example, human keratinous fibers such as hair, comprising, in a medium suitable for dyeing:

(i) at least one oxidation dye precursor chosen from 1-(4-aminophenyl)pyrrolidines of formula (I) and acid addition salts thereof:

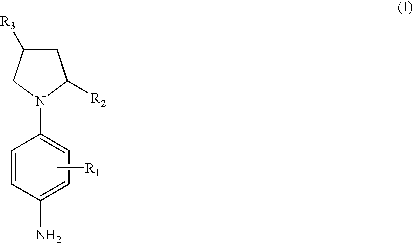

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups;

R₃ is chosen from a hydrogen atom, and a hydroxyl group; and

(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group.

Non-limiting examples of the acid addition salts of the 1-(4-aminophenyl)pyrrolidines of formula (I) which may be used in the dyeing compositions according to the invention are chosen from hydrochlorides, hydrobromides, sulfates, tartrates, lactates and acetates.

The dyeing composition in accordance with the invention leads, after combining with an oxidizing composition, to oxidation dyes with at least one of the following advantages: a rich palette of colors and shades, intense colors while minimizing degradation of the keratinous fibers, colors which tend to be not very selective, colors which are chromatic and aesthetically pleasing and colors which are resistant to at least one atmospheric agent such as light, adverse weather conditions, perspiration and various treatments to which the hair may be subjected.

Another embodiment of the invention relates to a ready-to-use composition for oxidation dyeing keratinous fibers comprising, in an medium suitable for dyeing:

(i) at least one oxidation dye precursor chosen from the 1-(4-aminophenyl)-pyrrolidines -pyrrolidines of formula (I) and acid addition salts thereof:

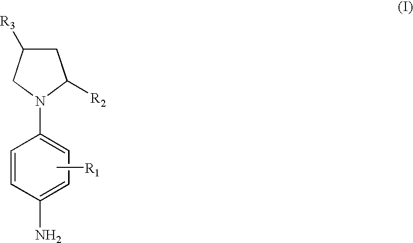

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups;

R₃ is chosen from a hydrogen atom, and a hydroxyl group; and

(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and

(iii) at least one oxidizing agent.

The expression ready-to-use composition is understood to mean, according to the purposes of the present invention, any composition intended to be applied immediately to the keratinous fibers, either stored as it is before use or obtained from the combining of two or more compositions.

The invention also relates to a method for oxidation dyeing keratinous fibers, for example, human keratinous fibers such as hair, comprising:

(1) applying to said fibers at least one composition (A) for oxidation dyeing of keratinous fibers comprising, in a medium suitable for dyeing:

(i) at least one oxidation dye precursor chosen from 1-(4-aminophenyl)pyrrolidines of formula (I) and acid addition salts thereof:

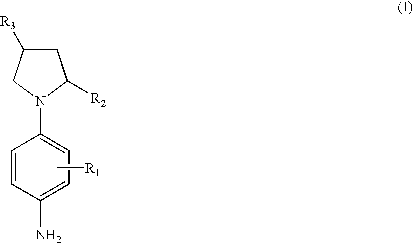

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups; and

R₃ is chosen from a hydrogen atom, and a hydroxyl group; and

(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a

nitrogen atom and a —CH— group; and

(2) developing a color by applying to said fibers a composition (B) comprising at least one oxidizing agent, wherein:

said at least one composition (B) is combined at the time of use with said at least one composition (A) or said at least one composition (B) is applied simultaneously with or immediately after, applying said at least one composition (A) without intermediate rinsing to said fibers.

The color may be developed at alkaline, neutral or acidic pH.

Another embodiment of the invention is a multi-compartment dyeing device, dyeing “kit” for oxidation dyeing keratinous fibers, for example, human keratinous fibers, such as hair.

Another embodiment of the invention is directed to a method for oxidation dyeing keratinous fibers;, for example, human keratinous fibers such as hair, comprising:

(1) applying to said fibers at least one composition (A) for oxidation dyeing of keratinous fibers comprising, in a medium suitable for dyeing:

(i) at least one oxidation dye precursor chosen from 1-(4-aminophenyl)pyrrolidines of formula (I) and acid addition salts thereof:

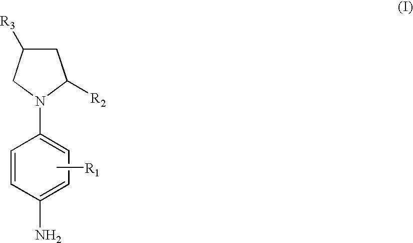

wherein:

R₁ is chosen from a hydrogen atom, (C₁-C₆)kyl groups, (C₁-C₅)mnohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups; and

R₃ is chosen from a hydrogen atom, and a hydroxyl group; and

(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and

(2) developing a color by applying to said fibers at least one composition (B) comprising at least one oxidizing agent, wherein said at least one composition (B) is combined at the time of use with said at least one composition (A) to form a combination;

(3) leaving said combination on said fibers for a time ranging, for example, from 1 to 60 minutes, such as, for example, from 10 to 40 minutes;

(4) rinsing said fibers and optionally shampooing and optionally further rinsing said fibers; and

(5) drying said fibers.

Another embodiment of the invention is directed to a kit comprising at least two compartments, wherein:

(1) a first compartment comprises:

(i) at least one oxidation dye precursor chosen from 1-(4-aminophenyl)pyrrolidines of formula (I) and acid addition salts thereof:

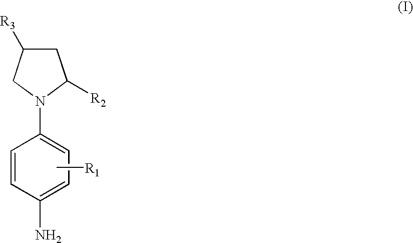

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups; and

R₃ is chosen from a hydrogen atom, and a hydroxyl group; and

(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and

(2) a second compartment comprising at least one oxidizing agent.

Another non-limiting embodiment of the invention is directed to a kit comprising at least three compartments, wherein:

(1) a first compartment comprises at least one oxidation dye precursor chosen from 1-(4-aminophenyl)pyrrolidines of formula (I) and acid addition salts thereof:

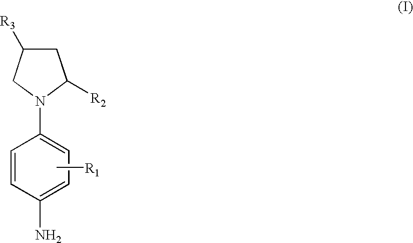

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups; and

R₃ is chosen from a hydrogen atom, and a hydroxyl group;

(2) a second compartment comprises at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and

(3) a third compartment comprises at least one oxidizing agent.

These examples are non-limiting and other aspects, embodiments and advantages of the invention will emerge more clearly upon reading the description and examples that follow, which are also intended to be non-limiting.

The at least one 1-(4-aminophenyl)pyrrolidines of formula (I) according to the invention are compounds known to persons skilled in the art, examples of which, are described and prepared in U.S. Pat. Nos. 5,851,237, 5,876,464 and 5,993,491, all of which are incorporated by reference herein

In one embodiment of the invention, said 1-(4-aminophenyl)pyrrolidines of formula (I) are chosen from such compounds wherein:

R₁, R₂ and R₃ are each a hydrogen atom. Thus the compound of formula (I) is then 1-(4-aminophenyl)pyrrolidine.

In another embodiment of the invention, said 1-(4-aminophenyl)pyrrolidines of formula (I) are chosen from such compounds wherein:

R₁ and R₃ are each a hydrogen atom and R₂ is a —CH₂OH group. Thus, the compound of formula (I) is 1-(4-aminophenyl)-2-pyrrolidinemethanol.

In yet another embodiment of the invention, said 1-(4-aminophenyl)pyrrolidines of formula (I) are chosen from such compounds wherein:

R₁ is a hydrogen atom, R₂ is a —CH₂OH group and R₃ is a hydroxyl group. Thus the compound of formula (I) is 1-(4-aminophenyl)-4-hydroxy-2-pyrrolidinemethanol.

In yet another embodiment of the invention, said 1-(4-aminophenyl)pyrrolidines of formula (I) are chosen from such compounds wherein:

R₁ and R₃ are each a hydrogen atom and R₂ is a —CONH₂ group. Thus the compound of formula (I) is N-(4-aminophenyl)prolineamide.

The at least one 1-(4-aminophenyl)pyrrolidines of formula (I) and acid addition salts thereof can be present in a composition according to the invention in an amount ranging from 0.001 to 10% by weight relative to the total weight of the composition in accordance with the invention such as, for example, 0.01 to 8% by weight relative to the total weight of the composition.

Among the direct nitrobenzene dyes which can be used according to the invention, there may be mentioned compounds of formula (II):

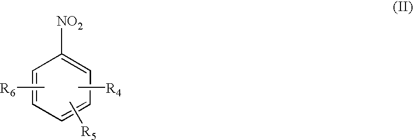

wherein:

R₄ is chosen from:

an amino radical optionally substituted with one or two groups chosen from (C₁-C₄)alkyl groups, (C₁-C₄)monohydroxyalkyl groups, (C₂-C₄)polyhydroxyalkyl groups, (C₁-C₄)aminoalkyl groups, mono((C₁-C₄)alkyl)amino((C₁-C₄)alkyl) groups, di((C₁-C₄)alkyl)amino((C₁-C₄)alkyl) groups, (C₁-C₄)ureidoalkyl groups, a hydroxyl group and aryl groups; and

aryl groups optionally substituted with at least one group chosen from a hydroxyl group, a carboxyl group, an amino group and di((C₁-C₄)alkyl)amino groups;

R₅ is chosen from:

a hydrogen atom; a hydroxyl group; (C₁-C₄)alkyl groups; (C₁-C₄)alkoxy groups; (C₁-C₄)monohydroxyalkyl groups; (C₁-C₄)polyhydroxyalkyl groups; (C₁-C₄)monohydroxyalkoxy groups; (C₂-C₄)polyhydroxyalkoxy groups; (C₁-C₄)aminoalkoxy groups; an amino radical optionally substituted with one or two groups chosen from (C₁-C₄)alkyl groups, (C₁-C₄)monohydroxyalkyl groups, (C₂-C₄)polyhydroxyalkyl groups, (C₁-C₄)aminoalkyl groups, mono((C₁-C₄)alkyl) amino((C₁-C₄)alkyl) groups, di((C₁-C₄)alkyl)amino((C₁-C₄)alkyl) groups, (C₁-C₄)ureidoalkyl groups and aryl groups; and aryl groups optionally substituted with at least one group chosen from a hydroxyl group, a carboxyl group, an amino group and di((C₁-C₄)alkyl)amino groups;

R₆ is chosen from: a hydrogen atom; a halogen atom; (C₁-C₄)alkyl groups; and a nitro group.

Examples of the direct nitrobenzene dyes of formula (II) above, are chosen from:

2-amino-4-methyl-5-N-(β-hydroxyethyl)aminonitrobenzene,

4-N-(β-ureidoethyl)aminonitrobenzene,

4-(N-ethyl-N-β-hydroxyethyl)amino-1-N-(β-hydroxyethyl)aminonitrobenzene,

2-N-(β-hydroxyethyl)amino-5-methylnitrobenzene,

5-chloro-3-N-(ethyl)amino-4-hydroxynitrobenzene,

5-amino-3-chloro-4-hydroxynitrobenzene,

2-N-(γ-hydroxypropyl)amino-5-N,N-bis(β-hydroxyethyl)aminonitrobenzene,

5-hydroxy-2-N-(γ-hydroxypropyl)aminonitrobenzene,

1,3-bis(β-hydroxyethyl)amino-4-chloro-6-nitrobenzene,

2,4-diaminonitrobenzene,

3,4-diaminonitrobenzene,

2,5-diaminonitrobenzene,

3-amino-4-hydroxynitrobenzene,

4-amino-3-hydroxynitrobenzene,

5-amino-2-hydroxynitrobenzene,

2-amino-5-hydroxynitrobenzene,

4-amino-3-hydroxynitrobenzene,

5-amino-2-hydroxynitrobenzene,

2-amino-3-hydroxynitrobenzene,

2-amino-5-N-(β-hydroxyethyl)amninonitrobenzene,

2-amino-5-N,N-bis(βhydroxyethyl)aminonitrobenzene,

2,5-N,N′-bis(βhydroxyethyl)aminonitrobenzene,

2-N-(β-hydroxyethyl)amino-5-N,N-bis(β-hydroxyethyl)aminonitrobenzene,

2-amino-5-N-(methyl)aminonitrobenzene,

2-N-(methyl)amino-5-N,N-bis(β-hydroxyethyl)aminonitrobenzene,

2-N-(methyl)amino-5-(N-methyl-N-β-hydroxyethyl)aminonitrobenzene,

2,5-N,N′-(β-hydroxyethyl)aminonitrobenzene,

2-N-(β-hydroxyethyl)amino-5-hydroxynitrobenzene,

3-methoxy-4-N-(β-hydroxyethyl)aminonitrobenzene,

2-N-(methyl)amino-4-β-hydroxyethyloxynitrobenzene,

2-amino-3-methyinitrobenzene,

2-N-(β-hydroxyethyl)amino-5-aminonitrobenzene,

2-amino-4-chloro-5-N-(β-hydroxyethyl)aminonitrobenzene,

2-amino-4-methyl-5-N-(β-hydroxyethyl)aminonitrobenzene,

2-amino-4-methyl-5-N-(methyl)aminonitrobenzene,

2-N-(β-hydroxyethyl)amino-5-methoxynitrobenzene,

2-amino-5-β-hydroxyethyloxynitrobenzene,

2-N-(β-hydroxyethyl)aminonitrobenzene,

3-amino-4-N-(β-hydroxyethyl)aminonitrobenzene,

3-β-hydroxyethyloxy-4-N-(β-hydroxyethyl)aminonitrobenzene,

2-N-(methyl)amino-4-β,γ-dihydroxypropyloxynitrobenzene,

2-N-(β-hydroxyethyl)amino-5-β-hydroxyethyloxynitrobenzene,

2-N-(β-hydroxyethyl)amino-5-β,γ-dihydroxypropyloxynitrobenzene,

2-hydroxy-4-N-(β-hydroxyethyl)aminonitrobenzene,

2-N-(methyl)amino-4-methyl-5-aminonitrobenzene,

2-amino-4-isopropyl-5-N-(methyl)aminonitrobenzene,

2-N-(methyl)amino-5-(N-methyl-N-β,γ-dihydroxypropyl)aminonitrobenzene,

3-N-(β-hydroxyethyl)amino-4-N-(β-hydroxyethyl)aminonitrobenzene,

2-amino-4-methyl-5-N-(β,γ-dihydroxypropyl)aminonitrobenzene,

2-amino-4-methyl-5-hydroxynitrobenzene,

2-N-(β-hydroxyethyl)amino-4-N-(β-hydroxyethyl)aminonitrobenzene,

2-amino-5-N-(β-aminoethyl)aminonitrobenzene,

2-N-(β-aminoethyl)amino-5-metlhoxynitrobenzene,

2-N-(methyl)amino-5-N-(β-aminoethyl)aminonitrobenzene,

2-N-(β-aminoethyl)amino-4-N,N-(dimethyl)aminonitrobenzene,

3-amino-4-N-(β-aminoethyl)aminonitrobenzene,

2-amino-4-methyl-5-N-(β-aminoethyl)aminonitrobenzene,

2-N-(β-aminoethyl)amino-5-N,N-bis(β-hydroxyethyl)aminonitrobenzene,

3-β-aminoethyloxy-4-aminonitrobenzene,

2-N-(methyl)amino-5-(N-δ-amino-n-butyl)aminonitrobenzene,

2-N-(γ-amino-n-propyl)amino-5-N,N-(dimethyl)aminonitrobenzene,

3-methoxy-4-N-(β-aminoethyl)aminonitrobenzene,

2-N-(β-aminoethyl)amino-5-aminonitrobenzene,

2-amino-4-chloro-5-N-(β-aminoethyl)aminonitrobenzene,

2-N-(β-aminoethyl)amino-4-methoxynitrobenzene,

2-N-(β-aminoethyl)aminonitrobenzene,

2-N-(β-aminoethyl)amino-5-N-(β-aminoethyl)aminonitrobenzene,

2-N-(β-aminoethyl)amino-4-β-hydroxyethyloxynitrobenzene,

3-β-hydroxyethyloxy-4-N-(β-aminoethyl)aminonitrobenzene,

2-amino-5-aminoethyloxynitrobenzene,

3-hydroxy-4-N-(β-aminoethyl)aminonitrobenzene,

2-N-(β-aminoethyl)amino-5-β-hydroxyethyloxynitrobenzene,

2-N-(β-aminoethyl)amino-4-hydroxynitrobenzene,

[2-N-hydroxy-2-N-(β-hydroxyethyl)amino]-3-nitro-6benzyloxy-2-ethylamine, and

[2-N-hydroxy-2-N-(β-hydroxypropyl)amino]-3-nitro-6-benzyloxy-2-ethylamine.

In one embodiment of the invention, the nitrobenzene dyes of formula (II) may include:

2-amino-4-methyl-5-N-(β-hydroxyethyl)aminonitrobenzene,

4-N-(β-ureidoethyl)aminonitrobenzene,

4-(N-ethyl-N-β-hydroxyethyl)amino-1-N-(β-hydroxyethyl)aminonitrobenzene,

2-N-(β-hydroxyethyl)amino-5-methylnitrobenzene,

5-chloro-3-N-(ethyl)amino-4-hydroxynitrobenzene,

5-amino-3-chloro-4-hydroxynitrobenzene,

2-N-(γ-hydroxypropyl)amino-5-N,N-bis(β-hydroxyethyl)aminonitrobenzene,

5-hydroxy-2-N-(γ-hydroxypropyl)aminonitrobenzene,

1,3-bis(β-hydroxyethyl)amino-4-chloro-6-nitrobenzene,

3,4-diaminonitrobenzene,

2-amino-5-hydroxynitrobenzene,

2-amino-3-hydroxynitrobenzene,

2-amino-5-N-(β-hydroxyethyl)aminonitrobenzene,

2-amino-5-N,N-bis(β-hydroxyethyl)aminonitrobenzene,

2-N-(β-hydroxyethyl)amino-5-N,N-bis(β-hydroxyethyl)aminonitrobenzene,

2-N-(β-hydroxyethyl)amino-5-hydroxynitrobenzene,

2-N-(β-hydroxyethyl)amino-5-aminonitrobenzene,

2-N-(β-aminoethyl)amino-4-methoxynitrobenzene, and

2-N-(β-aminoethyl)amino-5-β-hydroxyethyloxynitrobenzene.

Examples of direct cationic dyes include compounds of formulae (III), (IV) and (V):

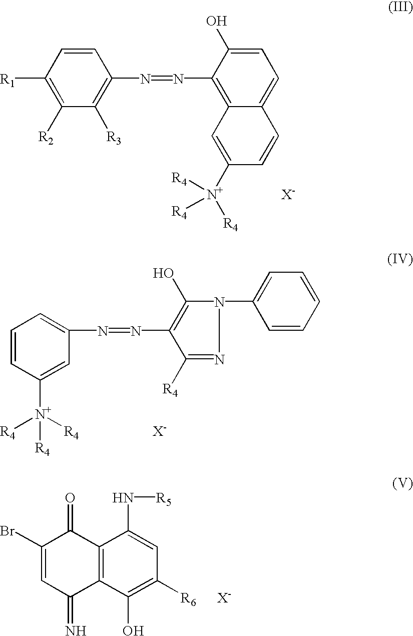

wherein:

R₁ is chosen from a hydrogen atom and an amino group;

R₂ is chosen from a hydrogen atom and a nitro group;

R₃ is chosen from a hydrogen atom, a nitro group and (C₁-C₄) alkoxy groups;

R₄ which are identical or different, are each chosen from (C₁-C₄)alkyl groups;

R₅ is chosen from a hydrogen atom and para-tri((C₁-C₄)alkyl) ammoniophenyl groups;

R₆ is chosen from a bromine atom and NH-para-tri((C₁-C₄)alkyl) aminophenyl groups; and

Examples of X⁻ include anions chosen from a chloride anion, a methylsulfate anion and an acetate anion.

One embodiment of the invention may employ the mesomeric forms of the compounds of formulae (III), (IV) and (V).

Examples of the compounds of formula (III) may include the dyes: Basic Brown 16, Basic Red 76, Basic Brown 17 and Basic Red 118.

Examples of the compounds of formula (IV) may include the dye, Basic Yellow 57.

Examples of the compounds of formula (V) may include the dye, Basic Blue 99.

The above-mentioned Color Index names include, for example, the following chemical structures (in the form of chlorides):

8-[(4-aminophenyl)azo]-7-hydroxy-2-trimethylammonionaphthalene,

8-[(2-methoxyphenyl)azo]-7-hydroxy-2-trimethylammonionaphthalene,

8-[(4-amino-3-nitrophenyl)azo]-7-hydroxy-2-trimethylammonionaphthalene,

8-[(4-amino-2-nitrophenyl)azo]-7-hydroxy-2-trimethylammonionaphthalene,

3-[(3-methyl-5-hydroxy-1-phenyl-1H-pyrazol-4-yl)azo]trimethylammoniobenzene,

3-[(4-amino-6-bromo-5,8-dihydro-1-hydroxy-8-imino-5-oxo-2-naphthalenyl)amino]trimethylammoniobenzene,

3-[(3,7-dibromo-5,8-dihydro-4-hydroxy-5-imino-8-oxo-1-naphthalenyl)amino]trimethylammoniobenzene.

The said compounds of formulae (III), (IV) and (V) may be present alone or in combination in the following commercially available products manufactured by WARNER JENKINSON chosen from: ARIANOR MAHOGANY, ARIANOR STEEL BLUE, ARIANOR MADDER RED, ARIANOR SIENNA BROWN, ARIANOR STRAW YELLOW, and ARIANOR BORDEAUX.

In another embodiment of the invention, the cationic direct dyes may be chosen from formulae (VI), (VII), (VIII), (VIII′) and (IX) below:

a) Compounds of Formula (VI)

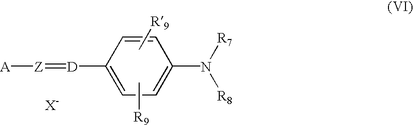

wherein:

Z and D, which are identical or different, are each chosen from a nitrogen atom and a —CH— group;

R₇ and R₈, which are identical or different, are each chosen from a hydrogen atom, a 4′-aminophenyl group, and C₁-C₄ alkyl groups, wherein said (C₁-C₄)alkyl groups are optionally substituted with a group chosen from a —CN group, a hydroxyl group and an —NH₂ group, and optionally wherein one of said R₇ and said R₈, together with a carbon atom of the benzene ring of formula (VI), forms a nitrogenous heterocycle optionally substituted with at least one group chosen from (C₁-C₄)alkyl groups, wherein said nitrogenous heterocycle optionally further comprises at least one heteroatom chosen from an oxygen atom and a nitrogen atom;

R₉ and R′₉, which are identical or different, are each chosen from a hydrogen atom, a halogen atom chosen from chlorine, bromine, iodine and fluorine, a cyano group, (C₁-C₄)alkyl groups, (C₁-C₄) alkoxy groups and acetyloxy groups;

X⁻ is an anion; and

A chosen from groups of formulae A1 A2, A3, A4, A5, A6, A7, A8, A9, A10, A11, A12, A13, A14, A15, A16, A17, A18 and A19:

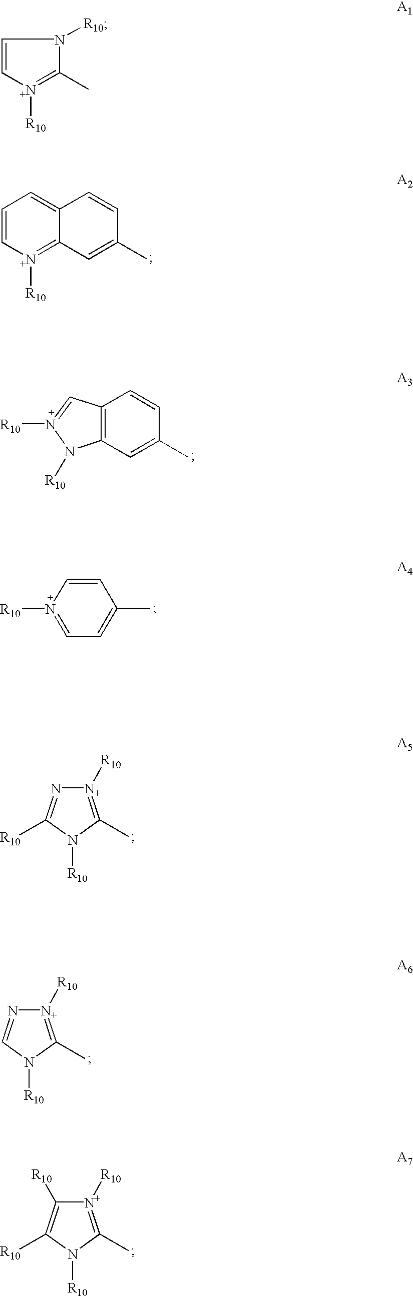

wherein:

R₁₀ is the same or different and each is chosen from (C₁-C₄) alkyl groups optionally substituted with at least one hydroxyl radical; and

R₁₁ is chosen from (C₁-C₄) alkoxy groups.

In one embodiment of the invention, the anion, X⁻ of formula (VI) is chosen from a chloride anion, a methyl sulfate anion, and an acetate anion.

b) Compounds of Formula (VII):

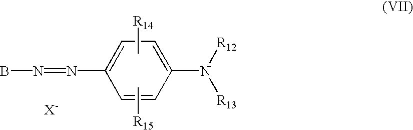

wherein:

R₁₂ is chosen from a hydrogen atom, and (C₁-C₄)alkyl groups;

R₁₃ is chosen from a hydrogen atom, a 4′-aminophenyl group, and (C₁-C₄)alkyl groups, wherein said (C₁-C₄) alkyl groups are optionally substituted with a group chosen from a —CN group and an amino group, and optionally wherein said R₁₃, together with said R₁₂, forms a nitrogenous heterocycle optionally substituted with at least one group chosen from (C₁-C₄)alkyl groups, wherein said nitrogenous heterocycle optionally further comprises at least one heteroatom chosen from an oxygen atom and a nitrogen atom;

R₁₄ and R₁₅, which are identical or different, are each chosen from a hydrogen atom, a halogen atom chosen from bromine, chlorine, iodine and fluorine, (C₁-C₄) alkyl groups, (C₁-C₄) alkoxy groups, and a —CN group;

X⁻ is an anion;

B is chosen from groups of formulae B1, B2, B3, B4, B5 and B6:

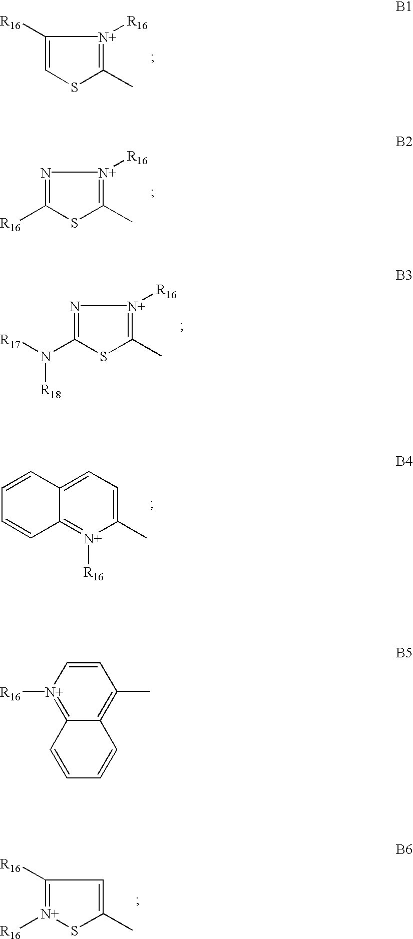

wherein:

R₁₆ is identical or different and each is chosen from (C₁-C₄)alkyl groups,

R₁₇ and R₁₈, which are identical or different, are each chosen from a hydrogen atom and (C₁-C₄)alkyl groups.

In one embodiment of the invention, the anion X⁻ of formula (VI) is chosen from a chloride anion, a methylsulfate anion and an acetate anion.

c) Compounds of Formulae (VIII) and (VIII′):

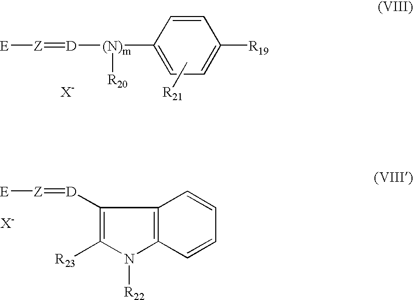

wherein:

Z and D₁ which are identical or different, are each chosen from a nitrogen atom and a —CH—group;

R₁₉ is chosen from a hydrogen atom, (C₁-C₄)alkoxy groups, an amino group, and a halogen atom chosen from bromine, chlorine, iodine and fluorine;

R₂₀ is chosen from a hydrogen atom, and (C₁-C₄)alkyl groups and optionally, wherein said R₂₀, together with a carbon atom of the benzene ring forms a nitrogenous heterocycle, wherein said nitrogenous heterocycle is optionally substituted with at least one group chosen from (C₁-C₄)alkyl groups, and optionally, wherein said nitrogenous heterocycle further comprises an member, wherein said member is an oxygen atom;

R₂₁ is chosen from a hydrogen atom and a halogen atom chosen from bromine, chlorine, iodine and fluorine;

R₂₂ and R₂₃, which are identical or different, are each chosen from a hydrogen atom and (C₁-C₄)alkyl groups;

m is an integer equal to 0 or 1;

X⁻ is an anion;

E is chosen from groups of formulae E1, E2, E3, E4, E5, E6, E7, and E8:

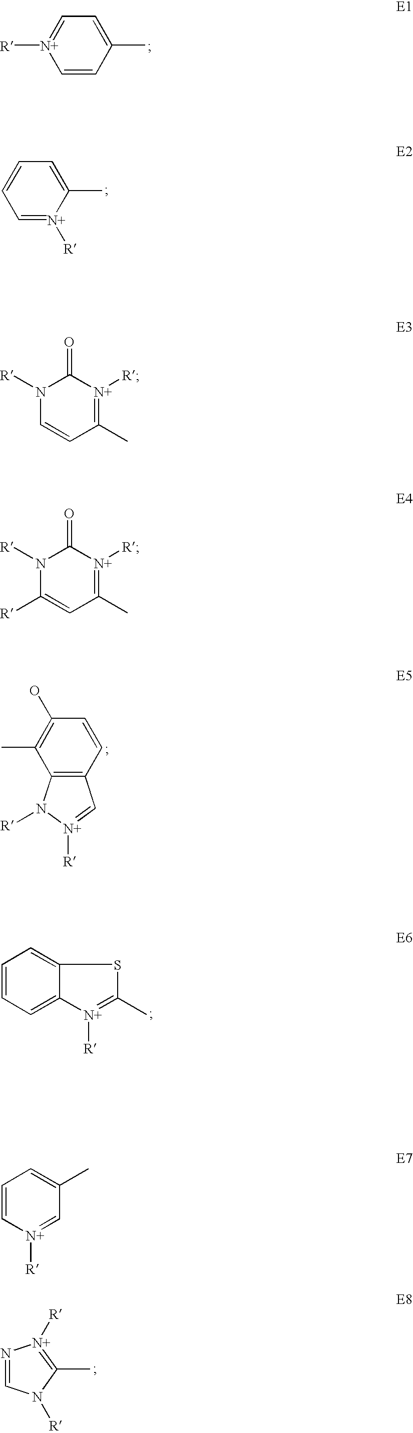

wherein:

R′ is identical or different and each is chosen from (C₁-C₄)alkyl groups; provided that:

when m is equal to 0 and D is a nitrogen atom, then E is a group of formula E9:

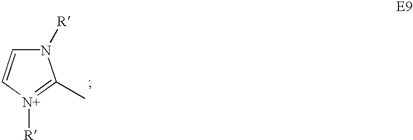

wherein:

R′ is identical or different and each is chosen from (C₁-C₄)alkyl groups.

In one embodiment of the invention, the anion, X⁻ of formula (VI) is chosen from a chloride anion, a methylsulfate anion and an acetate anion.

d) Compounds Formula (IX):

G—N═N—J (IX)

wherein:

G is chosen from groups of formulae G₁, G₂ and G₃:

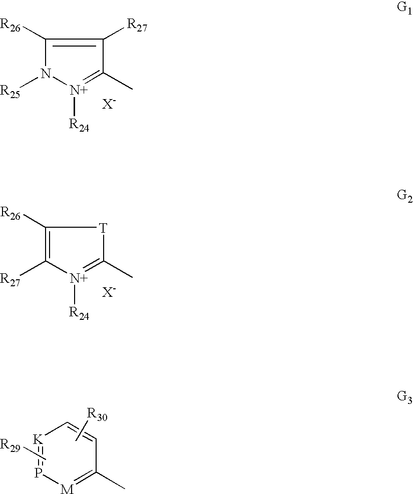

wherein:

R₂₄ is chosen from (C₁-C₄)alkyl groups, a phenyl group, wherein said phenyl group is optionally substituted with a group chosen from (C₁-C₄)alkyl groups, and a halogen atom chosen from chlorine, bromine, iodine and fluorine;

R₂₅ is chosen from (C₁-C₄)alkyl groups and a phenyl group;

R₂₆ is chosen from (C₁-C₄)alkyl groups, a hydrogen atom and a phenyl group;

R₂₇ is chosen from (C₁-C₄)alkyl groups and a phenyl group; provided that:

when said R₂₆ is other than a hydrogen atom, R₂₆ and R₂₇ optionally form a benzene ring, wherein said benzene ring is optionally substituted with at least one group chosen from (C₁-C₄)alkyl groups, (C₁-C₄)alkoxy groups, and a NO₂ group;

T is chosen from an oxygen atom, a sulfur atom and a group —NR₂₅, wherein R₂₅ is defined as above;

M is chosen from a —CH group, a —CR group, wherein R is chosen from (C₁-C₄)alkyl groups, and —N⁺R₂₈(X⁻)ᵣ groups, wherein R₂₈ is chosen from an O⁻, (C₁-C₄)alkoxy groups, and (C₁-C₄)alkyl groups and r is an integer equal to 0 or 1;

K is chosen from a —CH group, a —CR group, wherein R is chosen from (C₁-C₄)alkyl groups, and —N⁺R₂₈(X⁻)ᵣ groups, wherein R₂₈ is chosen from an O⁻, (C₁-C₄)alkoxy groups, and C₁-C₄ alkyl groups and r is an integer equal to 0 or 1;

P is chosen from a —CH group, a —CR group, wherein R is chosen from (C₁-C₄)alkyl groups; and —N⁺R₂₈(X⁻)ᵣ groups, wherein R₂₈ is chosen from an O⁻, (C₁-C₄)alkoxy groups, and (C₁-C₄)alkyl groups and r is an integer equal to 0 or 1;

R₂₉ and R₃₀, which are identical or different, are each chosen from a hydrogen atom, a halogen atom chosen from chlorine, bromine, iodine and fluorine, (C₁-C₄)alkyl groups, (C₁-C₄)alkoxy groups and an —NO₂ group;

X⁻ is an anion;

J is chosen from:

(a) a group of formula J₁:

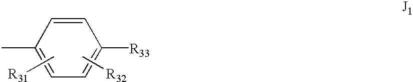

wherein:

R₃₁ is chosen from a hydrogen atom, a halogen atom chosen from chlorine, bromine, iodine and fluorine, (C₁-C₄)alkyl groups, (C₁-C₄)alkoxy groups, a hydroxyl group, an —NO₂ group, —NHR₃₄ groups, —NR₃₅R₃₆ groups and —NHCO(C₁-C₄)alkyl groups, wherein said R₃₄, said R₃₅, and said R₃₆ are defined below;

R₃₂ is chosen from a hydrogen atom, a halogen atom chosen from chlorine, bromine, iodine and fluorine, (C₁-C₄)alkyl groups, and (C₁-C₄)alkoxy groups;

R₃₃ is chosen from a hydrogen atom, a hydroxyl group, —NHR₃₄ groups, and —NR₃₅R₃₆ groups, wherein said R₃₄, said R₃₅, and said R₃₆ are defined below;

R₃₄ is chosen from a hydrogen atom, (C₁-C₄)alkyl groups, (C₁-C₄)monohydroxyalkyl groups, (C₂-C₄)polyhydroxyalkyl groups and a phenyl group;

R₃₅ and R₃₆, which are identical or different, are each chosen from (C₁-C₄)alkyl groups, (C₁-C₄)monohydroxyalkyl groups and (C₂-C₄)polyhydroxyalkyl groups;

wherein:

said R₃₁ and said R₃₂ optionally form a 5- or 6-membered ring, wherein said 5- or 6-membered ring optionally comprises at least one heteroatom chosen from a nitrogen atom, an oxygen atom, and a sulfur atom; and

said R₃₂ and one of said R₃₃ or said R₃₄ optionally form a 5- or 6-membered ring, wherein said 5- or 6-membered ring optionally comprises at least one heteroatom chosen from a nitrogen atom, an oxygen atom, and a sulfur atom;

(b) a 5- or 6-membered nitrogenous heterocyclic group optionally comprising at least one unit chosen from heteroatoms and carbonyl-containing groups, wherein said 5- or 6-membered nitrogenous heterocyclic group is optionally substituted with at least one group chosen from (C₁-C₄) alkyl groups, an amino group, and a phenyl group.

In one embodiment of the invention, said 5- or 6-membered nitrogenous heterocyclic group of (b) as defined above is chosen from a group of formula J₂:

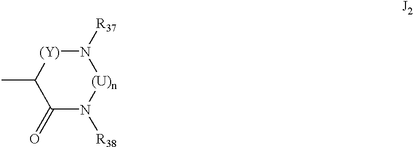

wherein:

R₃₇ and R₃₈, which are identical or different, are each chosen from a hydrogen atom, (C₁-C₄)alkyl groups and a phenyl group;

Y is chosen from a group —CO—and a group

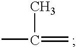

n is an integer equal to 0 or 1; provided that:

when n is equal to 1, then U is a —CO—group.

In another embodiment of the invention, mesomeric forms of the cationic direct dyes chosen from formulae (VI), (VII), (VIII), (VIII′) and (IX) may be utilized.

In yet another embodiment of the invention, in structures of formulae (VI), (VII), (VIII), (VIII′) and (IX) as in structures (III), (IV) and (V) as defined above, said (C₁-C₄)alkoxy groups can, for example be chosen from a methoxy group and an ethoxy group; and said alkyl groups can, for example, be chosen from a methyl group, an ethyl group, and a butyl group.

The cationic dyes of formulae (VI), (VII), (VIII) and (VIII′) are compounds described, for example, in Patent Applications WO 95/15144, WO 95/01772 and EP-A 714954; those of formula (IX) are compounds which are described, for example, in Patent Applications FR-2189006, FR-2285851 and FR-2140205 and its certificates of addition. All the above mentioned patents and applications are incorporated by reference, herein.

Examples of the cationic direct dyes of formula (VI) which can be used in the dyeing compositions in accordance with the invention, include, for example, the compounds of formula (VI₁) to (VI₅₄):

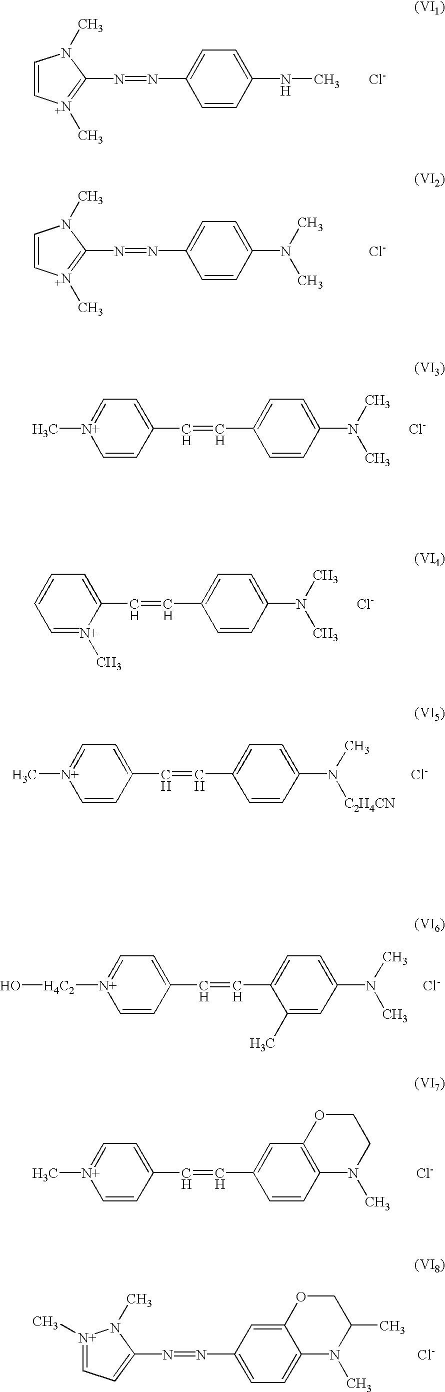

In one embodiment of the invention, the cationic direct dyes of formula (IV) can be chosen, for example, from the compounds of formulae (VI₁), (VI₂), (VI₁₄) and (VI₃₁).

In yet another embodiment of the invention, the cationic direct dyes of formula (VII) can be chosen, for example, from the compounds of formulae (VII₁) to (VII₉):

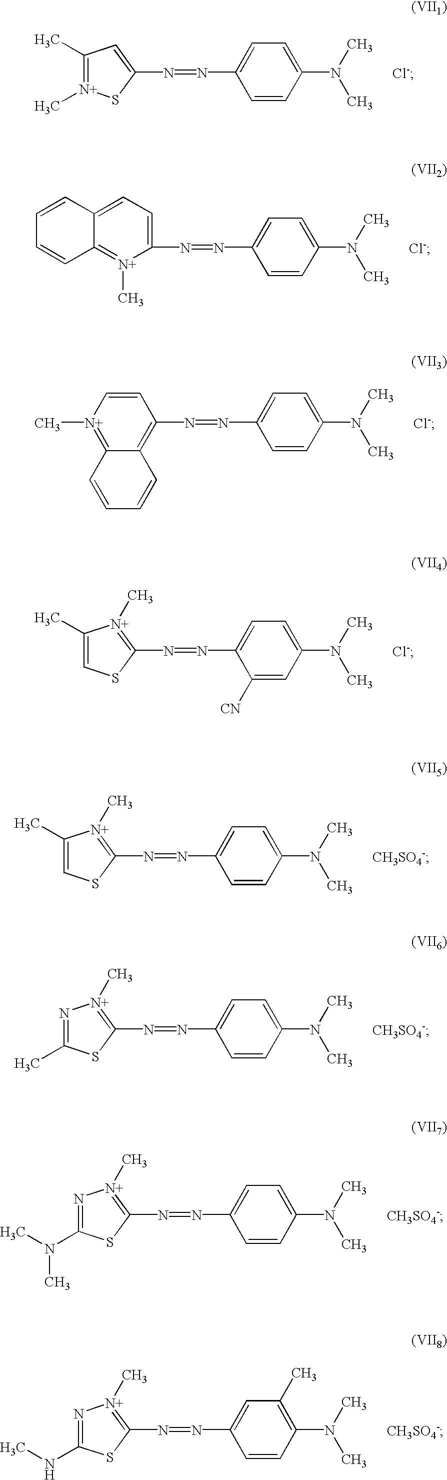

The cationic direct dyes of formula (VIII) which can be used in the dyeing compositions in accordance with the invention, may be, for example, chosen from compounds of formulae (VIII₁) to (VIII₁₈):

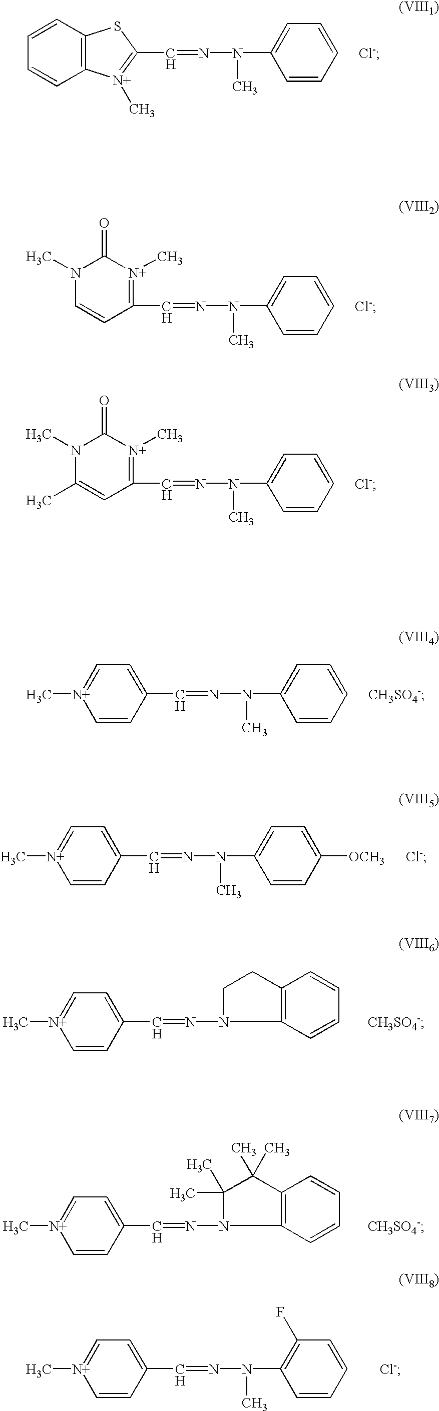

In one embodiment of the invention the cationic direct dyes of formula (VIII) can be chosen from, for example, compounds of formulae(VIII₄), (VIII₅) and (VIII₁₃).

In yet another embodiment of the invention, the cationic direct dyes of formula (VIII′), which may be used in the dyeing compositions according to the invention, can be chosen from, for example, compounds of formulae (VIII′₁) to (VIII′₃):

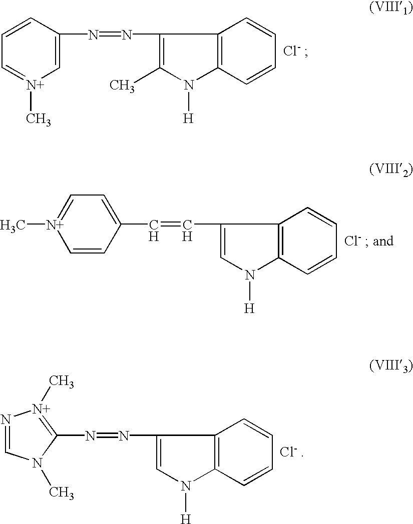

Among the cationic direct dyes of formula (IX) which can be used in the dyeing compositions in accordance with the invention are compounds chosen from, for example, compounds of formulae (IX₁) to (IX₇₇):

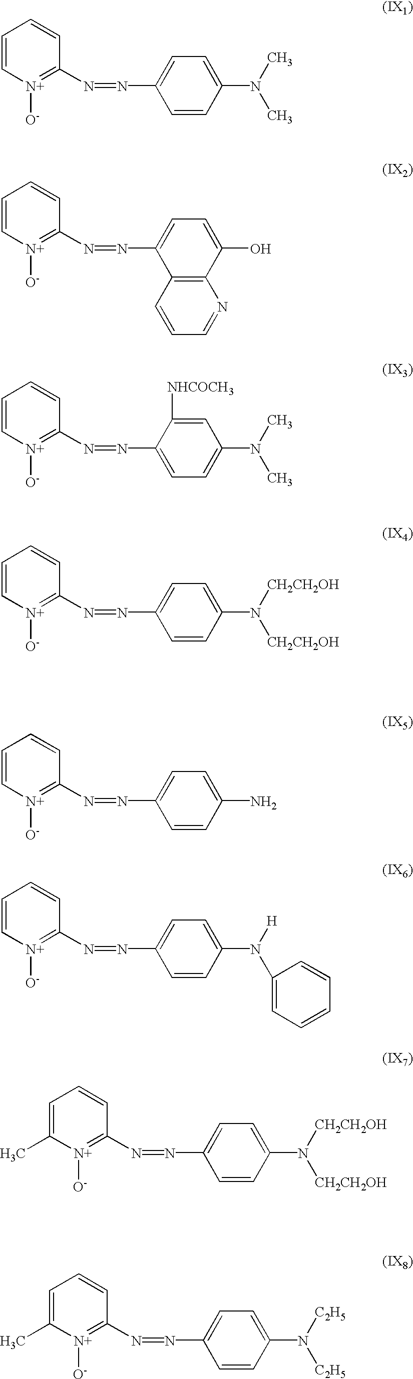

The at least one direct dye chosen from nitrobenzene direct dyes and cationic direct dyes according to the invention may be present in a composition of the invention in an amount ranging, for example, from 0.001 to 20% by weight relative to the total weight of the composition, such as, from 0.005 to 10% by weight relative to the total weight of the composition.

In another embodiment, the compositions of the invention further comprise at least one coupler. Examples of the at least one coupler include: meta-phenylenediamines, meta-aminophenols, meta-diphenols, naphthols and heterocyclic couplers, such as, indole derivatives, indoline derivtives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and acid addition salts thereof.

In one embodiment, the at least one coupler is chosen from: 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole,2,6-dimethylpyrazolo[1,5-b]-1,2,4-triazole, and acid addition salts of any of the foregoing compounds.

In one embodiment, said at least one coupler may be present in said composition according to the invention in an amount ranging, for example, from 0.0001 to 15% by weight relative to the total weight of the composition.

The dyeing composition in accordance with the invention may further comprise at least one additional oxidation base which is different from the 1-(4-aminophenyl)pyrrolidines of formula (I) and their acid addition salts, the at least one direct dye of the present invention.

In one embodiment, the at least one additional oxidation base which may be used according to the invention, may be chosen, for example, from para-phenylenediamine, para-tolylenediamine, 2-hydroxyethyl-para-phenylenediamine, 1-N,N-bis(2-hydroxyethyl)-para-phenylenediamine, para-aminophenols, such as, 3-methyl-4-aminophenol and 4-aminophenol, ortho-phenylene diamines, ortho-aminophenols, double bases, heterocyclic bases, such as, pyrimidines, such as, 2,4,5,6-tetraaminopyrimidine and pyrazoles, such as, 11-(2-hydroxyethyl)-4,5-diaminopyrazole.

The at least one additional oxidation base may be present in the composition according to the invention in an amount ranging, for example, from 0.0001 to 15% by weight relative to the total weight of said composition.

The appropriate dyeing medium for the composition according to the invention can be, for example, an aqueous medium comprising water and may advantageously comprise at least one cosmetically acceptable organic solvent. The at least one cosmetically acceptable organic solvent may, for example, be chosen from alcohols, such as ethyl alcohol, isopropyl alcohol, benzyl alcohol and phenylethyl alcohol, chosen from glycols(for example, ethyleneglycol, propyleneglycol, butyleneglycol, dipropyleneglycol and diethyleneglycol) and ethers of glycols(for example, monomethyl, monoethyl and monobutyl ethers of ethyleneglycol and for example, monomethyl ether of propyleneglycol and alkyl ethers of diethyleneglycol, such as, for example, monoethylether and monobutylether of diethyleneglycol).

Said at least one cosmetically acceptable organic solvent may be present in the composition according to the invention in an amount ranging from, for example, 1 to 40% by weight relative to the total weight of the composition.

The composition according to the invention may further comprise an effective quantity of at least one agent, moreover previously known in oxidation dyeing, such as various customary adjuvants, for example, sequestrants such as EDTA and etidronic acid, UV-screening agents, waxes, cyclic and linear, branched and unbranched, organomodified (in particular with amine groups) or otherwise, volatile and nonvolatile silicones, preservatives, ceramides, pseudoceramides, vegetable oils, mineral oils and synthetic oils, vitamins and provitamins, such as, panthenol, opacifiers, thickening agents, such as, crosslinked polyacrylic acids and hydroxyalkyl celluloses, and the like, and cationic polymers.

Cationic Polymers

As used herein, “cationic polymer” refers to polymers chosen from polymers comprising at least one cationic group and polymers comprising at least one group which can be ionized to form cationic groups.

Representative cationic polymers which may be used in accordance with the present invention include any of those already known to improve at least one cosmetic property of hair, such as, for example, those described in patent application EP-A-0 337 354 and in French patent applications FR-A-2 270 846, 2 383 660, 2 598 611, 2 470 596 and 2 519 863, the disclosures of which are incorporated herein by reference.

According to the present invention, the at least one cationic polymer may be chosen from polymers comprising at least one unit, wherein said at least one unit comprises at least one group chosen from primary amine groups, secondary amine groups, tertiary amine groups and quaternary amine groups, wherein said at least one group forms part of the polymer skeleton, or is carried by at least one lateral substituent on said polymer skeleton.

According to the present invention, the at least one cationic polymer has a number-average molecular mass generally ranging for example from 500 to 5×10⁶, such as from 1×10³ to 3×10⁶.

The at least one cationic polymer may, for example, be chosen from polymers of quaternary polyammonium type, polymers of polyamino amide type and polymers of polyamine type. Such types of polymers are known in the art. They are for example described in French patents Nos. 2,505,348 and 2,542,997, the disclosures of which are incorporated by reference herein.

Non-limiting examples of cationic polymers include:

(1) homo- and co-polymers derived from at least one monomer chosen from acrylic esters, methacrylic esters and amides, wherein said homo- and co-polymers comprise at least one unit chosen from units of formulae: (I), (II), (III) and (IV):

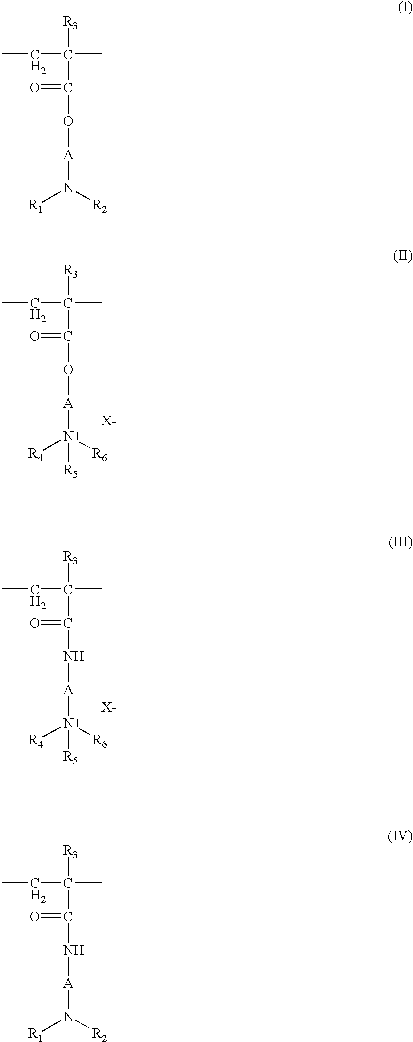

wherein:

R₃, which may be identical or different, are each chosen from a hydrogen atom and a methyl group;

A, which may be identical or different, are each chosen from linear and branched divalent (C₁-C₆)alkyl groups, such as,(C₂-C₃)alkyl groups, and (C₁-C₄)hydroxyalkyl groups;

R₄, R₅ and R₆, which may be identical or different, are each chosen from (C₁-C₁₈)alkyl groups, such as, (C₁-C₆), and a benzyl group;

R₁ and R₂, which may be identical or different, are each chosen from a hydrogen atom and (C₁-C₆)alkyl groups, such as, a methyl group and an ethyl group;

X⁻ is an anion chosen from anions derived from at least one inorganic acid and anions derived from at least one organic acid, such as a methylsulfate anion and halides, such as a chloride and a bromide.

Copolymers of family (1) may further comprise at least one unit derived from at least one comonomer chosen from vinyllactams, vinyl esters, acrylamides, methacrylamides, diacetone acrylamides, acrylamides and methacrylamides substituted on the nitrogen with at least one group chosen from (C₁-C₄) alkyls, acrylic acids, methacrylic acids, acrylic esters, and methacrylic esters. Non-limiting examples of vinyllactams include vinylpyrrolidone and vinylcaprolactam.

Non-limiting examples of copolymers of family (1) include:

copolymers derived from at least one monomer of (i) acrylamide and (ii) dimethylaminoethyl methacrylate quaternized with at least one group chosen from a dimethylsulfate group and dimethylhalides, such as the product sold under the name HERCOFLOC by the company Hercules;

copolymers derived from at least one monomer of (i) acrylamide and (ii) methacryloyloxyethyltrimethylammonium chloride described, for example, in patent application EP-A-080 976, the disclosure of which is incorporated herein by reference, and which is sold under the name BINA QUAT P 100 by the company Ciba Geigy;

copolymers derived from at least one monomer of (i) acrylamide and (ii) methacryloyloxyethyltrimethylammonium methosulfate, such as, for example, copolymers sold under the name RETEN by the company Hercules;

quaternized and non-quaternized vinylpyrrolidone/dialkylaminoalkyl acrylate copolymers and quaternized and non-quaternized vinylpyrrolidone/dialkylaminoalkyl methacrylate copolymers, such as the products sold under the name “GAFQUAT” by the company ISP, such as, for example, “GAFQUAT 734” or “GAFQUAT 755” and the products known as “COPOLYMER 845, 958 and 937”. These polymers are described in detail in French patents 2 077 143 and 2 393 573, the disclosures of which are incorporated herein by reference;

dimethylaminoethyl methacrylate/vinylcaprolactam/vinylpyrrolidone terpolymers, such as the product sold under the name GAFFIX VC 713 by the company ISP;

vinylpyrrolidone/methacrylamidopropyidimethylamine copolymers, such as the product sold under the name STYLEZE CC 10 by ISP; and

quaternized vinylpyrrolidone/dimethylaminopropylmethacrylamide copolymers, such as the product sold under the name “GAFQUAT HS 100” by the company ISP;

(2) cellulose ether derivatives comprising quaternary ammonium groups, such as those described in French patent 1,492,597, the disclosure of which is incorporated herein by reference, and polymers sold under the names “JR” (JR 400, JR 125 and JR 30M) and “LR” (LR 400, or LR 30M) by the company Union Carbide Corporation. These polymers are also defined in the CTFA dictionary as quaternary ammoniums of hydroxyethylcellulose which have reacted with an epoxide substituted with a trimethylammonium group;

(3) cationic cellulose derivatives such as cellulose copolymers and cellulose derivatives grafted with at least one water-soluble monomer of quaternary ammonium, such as those described in U.S. Pat. No. 4,131,576, the disclosure of which is incorporated herein by reference, such as hydroxyalkylcelluloses (such as, for example, hydroxymethylcelluloses, hydroxyethylcelluloses and hydroxypropylcelluloses, wherein said hydroxyalkylcelluloses are grafted with at least one salt chosen from, for example, methacryloylethyltrimethylammonium salts, methacrylamidopropyltrimethylammonium salts and dimethyldiallylammonium salts). For example, commercial products corresponding to the aforementioned cationic cellulose derivatives include the products sold under the names “CELQUAT L 200” and “CELQUAT H 100” by the company National Starch;

(4) cationic polysaccharides, such as those described in U.S. Pat. Nos. 3,589,578 and 4,031,307, the disclosures of which are incorporated herein by reference, such as guar gums comprising at least one cationic trialkylammonium group. For example, guar gums modified with at least one salt, such as a chloride salt, of 2,3-epoxypropyltrimethylammonium may be used in the present invention. Such products are sold in particular under the trade names JAGUAR C13 S, JAGUAR C 15, JAGUAR C 17 and JAGUAR C162 by the company Meyhall;

(5) polymers comprising (i) at least one piperazinyl unit and (ii) at least one group chosen from divalent alkylene groups and divalent hydroxyalkylene groups, wherein said at least one group optionally comprises at least one chain chosen from straight chains and branched chains, wherein said at least one chain is optionally interrupted by at least one entity chosen from an oxygen atom, a sulfur atom, a nitrogen atom, aromatic rings and heterocyclic rings, the oxidation products of said polymers and the quaternization products of said polymers. For example, such polymers are described in French patents 2,162,025 and 2,280,361, the disclosures of which are incorporated herein by reference;

(6) water-soluble polyamino amides which may be prepared by at least one polycondensation reaction of at least one acidic compound and at least one polyamine compound, wherein said polyamino amides may be crosslinked with at least one crosslinking agent chosen from epihalohydrins, diepoxides, dianhydrides, unsaturated dianhydrides, bis-unsaturated derivatives, bis-halohydrins, bis-azetidiniums, bis-haloacyldiamines, bis-alkyl halides and oligomers derived from reaction of at least one difunctional compound with at least one compound chosen from bis-halohydrins, bis-azetidiniums, bis-haloacyldiamines, bis-alkyl halides, epihalohydrins, diepoxides and bis-unsaturated derivatives, wherein said crosslinking agent may be used in a proportion generally ranging from 0.025 mol to 0.35 mol per amine group of said polyamino amide, wherein said polyamino amides may optionally be alkylated, and wherein if said polyamino amides comprise at least one tertiary amine group, said polyamino amides may optionally be quaternized. For example, such polymers are described in French patents 2,252,840 and 2,368,508, the disclosures of which are incorporated herein by reference;

(7) polyamino amide derivatives derived from condensation of at least one polyalkylene polyamine with at least one polycarboxylic acid, followed by alkylation with at least one bifunctional agent. Non-limiting examples of such polyamino amide derivatives include adipic acid/dialkylaminohydroxyalkyldialkylenetriamine polymers wherein said alkyl group is chosen from (C₁-C₄)alkyl groups, such as a methyl group, an ethyl group and a propyl group. For example, such polymers are described in French patent 1,583,363, the disclosure of which is incorporated herein by reference.

Other non-limiting examples of such derivatives include the adipic acid/dimethylaminohydroxypropyl/diethylenetriamine polymers sold under the name “CARTARETINE F, F4 or F8” by the company Sandoz.

(8) polymers derived from the reaction of (i) at least one polyalkylene polyamine comprising two primary amine groups and at least one secondary amine group with (ii) at least one dicarboxylic acid chosen from diglycolic acid and saturated aliphatic dicarboxylic acids comprising from 3 to 8 carbon atoms. According to the present invention, the molar ratio of the at least one polyalkylene polyamine to the at least one dicarboxylic acid generally ranges from 0.8:1 to 1.4:1. The polyamino amide resulting from the above reaction may be reacted with epichlorohydrin in a molar ratio of epichlorohydrin to the at least one secondary amine group of the polyamino amide generally ranging from 0.5:1 to 1.8:1. For example, such polymers are described in U.S. Pat. Nos. 3,227,615 and 2,961,347, the disclosures of which are incorporated herein by reference.

Polymers of this type are sold in particular under the name “HERCOSETT 57” by the company Hercules Inc. and under the name “PD 170” or “DELSETTE 101” by the company Hercules in the case of adipic acid/epoxypropyl/diethylenetriamine copolymers.

(9) cyclopolymers of alkyldiallylamine and cyclopolymers of dialkyidiallylammonium, such as hiomopolymers and copolymers comprising, as a constituent of the chain, at least one unit chosen from units of formulae (V) and (VI):

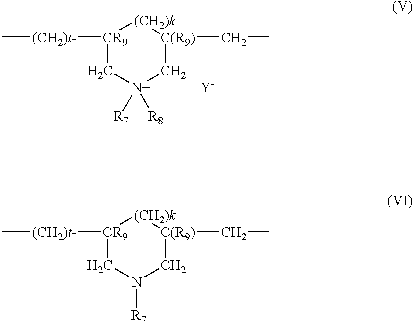

wherein:

k and t, which may be identical or different, are each chosen from 0 and 1, with the proviso that the sum of k+t is equal to 1;

R₉, which may be identical or different, are each chosen from a hydrogen atom and a methyl group;

R₇ and R₈, which may be identical or different, are each chosen from alkyl groups comprising from (C₁-C₂₂)alkyl groups, such as, (C₁-C₄)alkyl groups, hydroxyalkyl groups, such as (C₁-C₅)hydroxyalkyl groups, and (C₁-C₄)amidoalkyl groups;

R₇ and R₈, together with the nitrogen cation to which they are commonly bonded, may additionally form at least one cationic heterocyclic group, such as a cationic piperidyl group and a cationic morpholinyl group;

Y⁻ is an anion, such as a bromide anion, a chloride anion, an acetate anion, a borate anion, a citrate anion, a tartrate anion, a bisulfate anion, a bisulfite anion, a sulfate anion and a phosphate anion. For example, such polymers are described in French patent 2,080,759 and in its Certificate of Addition 2,190,406, the disclosures of which are incorporated herein by reference.

Non-limiting examples of the polymers defined above include the dimethyldiallylammonium chloride homopolymer sold under the name “MERQUAT 100” by the company Calgon (and its homologues of low weight-average molecular mass) and copolymers of diallyldimethylammonium chloride and of acrylamide, sold under the name “MERQUAT 550”.

(10) quaternary diammonium polymers comprising repeating units of formula (VII):

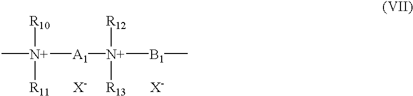

wherein:

R₁₀, R₁₁, R₁₂ and R₁₃, which may be identical or different, are each chosen from (C₁-C₂₀)aliphatic, (C₂-C₂₂)alicyclic groups, (C₅-C₂₀)arylaliphatic groups, and lower hydroxyalkyl groups; and

additionally at least two of said R₁₀, R₁₁, R₁₂ and R₁₃, together with the nitrogen cations to which they are attached, may form at least one cationic heterocycle optionally comprising an additional heteroatom other than nitrogen; and

additionally, R₁₀, R₁₁, R₁₂ and R₁₃, which may be identical or different, are each chosen from linear and branched (C₁-C₆)alkyl groups substituted with at least one group chosen from a nitrile group, ester groups, acyl groups, amide groups and groups chosen from groups of formulae —CO—O—R₁₇—D and —CO—NH—R₁₇—D wherein R₁₇ is chosen from alkylene groups and D is chosen from quaternary ammonium groups;

A₁ and B₁, which may be identical or different, are each chosen from linear and branched, saturated and unsaturated (C₂-C₂₀)polymethylene groups, wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups; and

X⁻ is an anion chosen from anions derived from inorganic acids and anions derived from organic acids; and

A₁, R₁₀ and R₁₂ may optionally form, together with the nitrogen cations to which they are attached, at least one cationic piperazine ring;

with the proviso that if A₁ is chosen from linear and branched, saturated and unsaturated (C₂-C₂₀)polymethylene groups and linear and branched, saturated and unsaturated hydroxy(C₂-C₂₀) groups, B₁ may also be chosen from groups of formula:

—(CH₂)ₙ—CO—D—OC—(CH₂)ₙ—

wherein:

n is a number between 1 and 100, such as, 1 and 50;

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂—; and

—[CH₂—CH(CH₃)—O]_{y}—CH₂—CH(CH₃)—;

wherein x and y, which may be identical or different, are each chosen from integers ranging from 1 to 4 i(in which case x and y represent a defined and unique degree of polymerization) and any number ranging from 1 to 4 (in which case x and y represent an average degree of polymerization);

b) bis-secondary diamine residues such as piperazine derivatives;

c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and

d) ureylene groups of formula: —NH—CO—NH—.

In one embodiment, X⁻ is an anion chosen from a chloride anion and a bromide anion.

According to the present invention, the quarternary diammonium polymers have a number-average molecular mass generally ranging, for example, from 1000 to 100,000.

For example, polymers of this type are described in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020, the disclosures of which are incorporated herein by reference.

Further, according to the present invention, polymers comprising repeating units of formula (VIII) may be used:

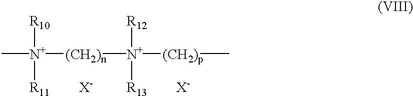

wherein:

R₁₀, R₁₁, R₁₂ and R₁₃, which may be identical or different, are each chosen from (C₁-C₄)alkyl groups and (C₁-C₄;)hydroxyalkyl groups;

n and p, which may be identical or different, are each chosen from integers ranging from 2 to 20; and

X⁻ is an anion chosen from anions derived from inorganic acids and anions derived from organic acids.

(11) polyquaternary ammonium polymers comprising repeating units of formula (IX):

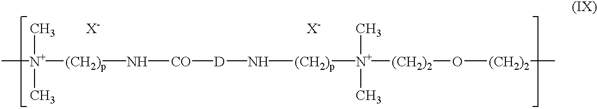

wherein:

p is an integer ranging from 1 to 6,

D is chosen from a direct bond and —(CH₂)ᵣ—CO—groups, wherein r is a number equal to 4 or 7, and

X⁻ is an anion chosen from anions derived from inorganic acids and anions derived from organic acids.

For example, such compounds are described in patent application EP-A-122,324, the disclosure of which is incorporated by reference herein, and may be prepared according to the procedures described in the U.S. Pat. Nos. 4,157,388, 4,390,689, 4,702,906, and 4,719,282, the disclosures of which are incorporated by reference herein.

Among these, there may be mentioned for example the products “Mirapol A 15”, “Mirapol AD1”, “Mirapol AZ1” and Mirapol 175” sold by the company Miranol.

(12) quaternary polymers of vinylpyrrolidone and quaternary polymers of vinylimidazole, such as, for example, the products sold under the names LUVIQUAT FC 905, FC 550 and FC 370 by the company BASF.

(13) polyamines, such as POLYQUART H sold by Henkel under the reference name “POLYETHYLENE GLYCOL (15) TALLOW POLYAMINE” in the CTFA dictionary.

(14) crosslinked (meth)acryloyloxy(C₁-C₄)alkyltri(C₁-C₄)alkylammonium salt polymers, such as the polymers derived from homopolymerization of dimethylaminoethyl methacrylate quaternized with methyl chloride and polymers derived from copolymerization, for example, of acrylamide with dimethylaminoethyl methacrylate quaternized with a methyl halide (such as methyl chloride), wherein the homo- or copolymerization is followed by crosslinking with at least one compound comprising olefinic unsaturation, such as methylenebisacrylamide. For example, a crosslinked acrylamide/methacryloyloxyethyltrimethylammonium chloride copolymer (20/80 by weight) in the form of a dispersion comprising about 50% by weight of said copolymer in mineral oil may be used. This dispersion is sold under the name “SALCARE SC 92” by the company Allied Colloids. Further, a crosslinked methacryloyloxyethyltrimethylammonium chloride homopolymer comprising about 50% by weight of the homopolymer in mineral oil or in a liquid ester may be used. These dispersions are sold under the names “SALCARE SC 95” and “SALCARE SC 96” by the company Allied Colloids.

The at least one cationic polymer according to the present invention, may for example, be chosen from polyalkyleneimines (such as polyethyleneimines), polymers comprising at least one vinylpyridine unit, polymers comprising at least one vinylpyridinium unit, condensates of polyamines, condensates of epichlorohydrin, quaternary polyureylenes and chitin derivatives.

Another embodiment of the invention comprises at least one cationic polymer chosen from polymers chosen from formulae (1), (9), (10), (11) and (14) above. Specifically, polymers of formulae (W) and (U) can be used:

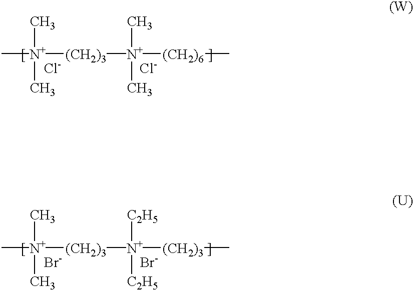

Generally, the at least one cationic polymer is present in the composition of the invention in an amount ranging, for example, from 0.01% to 10% by weight relative to the total weight of the final composition, such as, for example, from 0.05% to 5% by weight relative to the total weight of the final composition, and further still from 0.1% to 3% by weight relative to the total weight of the final composition.

The dyeing composition of the invention may further comprise at least one agent chosen from reducing agents and antioxidants. Said reducing agents and said antioxidants may be chosen from sodium sulfite, thioglycolic acid, thiolactic acid, sodium bisulfite, dehydroascorbic acid, hydroquinone, 2-methylhydroquinone, tert-butylhydroquinone and homogentisic acid.

In one embodiment of the invention, said at least one agent chosen from reducing agents and antioxidants may be present in the dyeing composition in an amount ranging, for example, from 0.05 to 1.5% by weight relative to the total weight of the dyeing composition.

The dyeing composition according to the invention may further comprise at least one fatty alcohol. Representatives of said at least one fatty alcohol that can be used according to the invention can include, for example, cetyl alcohol, stearyl alcohol, and oleyl alcohol. Said at least one fatty alcohol may be present in the dyeing composition according to the invention, in an amount ranging, for example, from 0.001 to 20% by weight relative to the total weight of the composition.

The dyeing composition of the invention may further comprise at least one surfactant chosen from nonionic, anionic, cationic and amphoteric surfactants. Said at least one surfactant may be present in the dyeing composition according to the invention, in an amount ranging, for example, from 0.1 to 20% by weight relative to the total weight of the composition.

In one embodiment of the invention, the dyeing composition comprises at least one surfactant chosen from nonionic surfactants.

One skilled in the art should take care to select said optionally complementary compounds, such that the advantageous properties intrinsically associated with the dye composition according to the invention are not, or are not substantially, adversely affected by the additions envisaged.

The at least one oxidizing agent according to the invention may, for example, be chosen from hydrogen peroxide, urea peroxide, alkali metal bromates, alkali metal ferricyanides, and persalts such as perborates and persulfates.

In one embodiment of the invention, the at least one oxidizing agent is hydrogen peroxide and said at least one oxidizing agent may, for example, comprise a solution of hydrogen peroxide with a titre ranging, for example, from 1 to 40 in volume, such as, for example, 5 to 40 in volume.

It is also possible to use as the at least one oxidizing agent at least one oxidation-reduction enzyme such as laccases, peroxidases and 2 electron oxidoreductases (such as uricase), if necessary, in the presence of their corresponding donor or corresponding cofactor.

The pH of the dyeing composition or of the ready-to-use composition applied to the keratinous fibers [composition resulting from the combination of the at least one dyeing composition according to the invention and of the at least one oxidizing agent], generally ranges, for example, from 3 to 12, such as, for example, from 6 to 11. The pH may be adjusted to the desired value by means of at least one agent chosen from acidifying agents and alkalinizing agents well known in the state of the art for dyeing keratinous fibers.

Representative alkalinizing agents, may include, for example, aqueous ammonia, alkali metal carbonates, alkanolamines such as mono-, di- and triethanolamines and derivatives thereof, oxyethylenated ethylenediamines and oxypropylenated ethylenediamines, hydroxyalkylamines, sodium hydroxide, potassium hydroxide and compounds of formula (X):

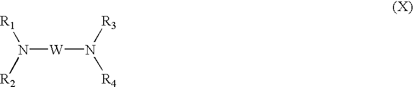

wherein:

W is a propylene residue optionally substituted with a group chosen from a hydroxyl group and (C₁-C₄)alkyl groups; and

R₁, R₂, R₃ and R₄, which are identical or different, are each chosen from a hydrogen atom, (C₁-C₄)alkyl groups and (C₁-C₄)hydroxyalkyl groups.

Representa itive acidifying agents include conventionally, by way of example, inorganic acids and organic acids such as hydrochloric acid, orthophosphoric acid, and carboxylic acids such as tartaric acid, citric acid, lactic acid and sulfonic acids.

The dyeing composition according to the invention may be provided in various forms, such as, liquids, powders, creams, gels, optionally pressurized, or in any other form suitable for dyeing keratinous fibers, such as human hair.

Unless otherwise indicated, all numbers expressing quantities of ingredients, properties such as molecular weight, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The following examples are intended to illustrate the invention without in anyway limiting the scope thereof.

### EXAMPLES

The following dyeing compositions were prepared: (expressed in grams)

| | | 2 |
|---|---|---|
| | 1 | prior |
| EXAMPLES | invention | art |
| 1-(4-Aminophenyl)pyrrolidine dihydrochloride | 0.235 | |
| (oxidation base according to the invention). | | |
| 2,4-Diamino-1-(β-hydroxyethyloxy)benzene | 0.241 | 0.241 |
| dihydrochloride (coupler) | | |
| N,N-bis(β-Hydroxyethyl)-para-phenylenediamine | | 0.196 |
| sulfate (oxidation base according to the prior art) | | |
| Cationic direct dye (VI₂) [Basic Red 51] | 0.168 | 0.168 |
| | AS* | AS* |
| Dye carrier (*) | qs | qs |
| Demineralized water qs | 100 | 100 |
| (*) Dye Carrier Composition | | |
| C₈-C₁₀ Alkyl polyglucoside in aqueous solution | 3.24 | g AS* |
| at 60%, sold under the name ORAMIX OG 110 ® | | |
| by the company SEPPIC | | |
| Ethanol | 18.0 | g |
| Benzyl alcohol | 1.8 | g |
| Polyethylene glycol 400 | 2.7 | g |
| Pentasodium salt of diethylenetriaminepentaacetic | 0.43 | g AS* |
| acid in aqueous solution at 40%, sold under the | | |
| name DISSOLUINE D-40 ® by the company | | |
| AKZO | | |
| Sodium metabissulfite | 0.205 | g |
| Aqueous ammonia containing 20.5% of NH₃ | 10.0 | g |

AS*denotes Active Substance

At the time of use, each of the dyeing compositions described above were mixed, weight for weight, with a solution of hydrogen peroxide at 20 volumes (6% by weight).

The mixtures thus prepared were applied for 30 minutes, to (1) locks of permanently waved natural grey hair which is 90% white, and (2) locks of natural grey hair which is 90% white. The locks were then rinsed, washed with a standard shampoo, rinsed again and then dried.

The color was then measured with a MINOLTA CM2002 colorimeter in the L*a*b* system.

In the L*a*b* system, the 3 parameters denote respectively the intensity (L*), the shade (a*) and the saturation (b*).

According to this system, the higher the value of L, the lighter and less intense the color. Conversely, the lower the value of L, the deeper or more intense the color.

a* and b* indicate two color axes, a* indicates the green/red color axis and b* the blue/yellow color axis. Values close to zero for a* and b* correspond to grey shades.

The selectivity of the color ΔE may be calculated by applying the following equation:

ΔE=\sqrt{(L*−L₀*)²+(a*−a₀*)²+(b*−b₀*)²}

In this equation, ΔE represents the difference in color between two locks, (in the present case the selectivity of the color), L*, a* and b* represent respectively the intensity, the shade and the saturation of the lock of natural hair dyed, L₀*, a₀* and b₀* represent respectively the intensity, the shade and the saturation of the lock of permanently waved hair dyed.

The higher the value of ΔE, the greater the difference in color between the two locks, and in the present case, the greater the selectivity of the dyeing.

The results have been grouped together in Table (I) below.

TABLE (I)

| | | | | | Selectivity |
|---|---|---|---|---|---|
| | | | | | (ΔE between |
| | | | | | natural and perma- |
| EXAMPLES | Type of hair | L* | a* | b* | nently waved hair) |
| 2 | natural | 38.25 | 10.81 | −2.72 | |
| 2 | permanently | 29.55 | 14.25 | −11.15 | 12.59 |
| | waved | | | | |
| 1 | natural | 20.36 | 6.96 | −9.34 | |
| 1 | permanently | 17.50 | 3.09 | −4.76 | 6.64 |
| | waved | | | | |

Conclusion

The dyeing with the combination according to the invention (1) is almost two times less selective than that of the prior art (2) [6.64 against 12.59].

The dyeing with the combination according to the invention (1) is more intense than that of the prior art (2) [lower L value].

What is claimed:

1. A composition for oxidation dyeing keratinous fibers, in a medium suitable for dyeing:
(i) at least one oxidation dye precursor chosen from 1-(4-aminophenyl)pyrrolidines of formula (I) and acid addition salts thereof: 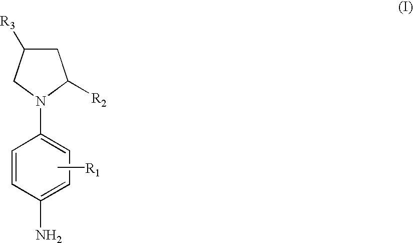
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, C₁-C₅ monohydroxyalkyl groups, and (C₂-C₅)polyhydroxyalkyl groups;
R₃ is chosen from a hydrogen atom, and a hydroxyl group; and
(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group.

2. A composition according to claim 1, wherein said R₁, said R₂ and said R₃ are each a hydrogen atom.

3. A composition according to claim 1, wherein said R₁ and said R₃ are each a hydrogen atom and said R₂ is a —CH₂OH group.

4. A composition according to claim 1, wherein said R₁ is a hydrogen atom, said R₂ is a —CH₂OH group and said R₃ is a hydroxyl group.

5. A composition according to claim 1, wherein said R₁ and said R₃ are each a hydrogen atom and said R₂ is a —CONH₂ group.

6. A composition according to claim 1, wherein said acid addition salts are chosen from hydrochlorides, hydrobromides, sulphates, tartrates, lactates and acetates.

7. A composition according to claim 1, wherein said at least one oxidation dye precursor chosen from the 1-(4-aminophenyl)pyrrolidines of formula (I) and acid addition salts thereof is present in said composition in an amount ranging from 0.001 to 10% by weight relative to the total weight of said composition.

8. A composition according to claim 7, wherein said at least one oxidation dye precursor chosen from the 1 -(4-aminophenyl)pyrrolidines of formula (I) and acid addition salts thereof is present in said composition in an amount ranging from 0.01 to 8% by weight of the total weight of said composition.

9. A composition according to claim 1, wherein said nitrobenzene dyes are chosen from compounds of formula (II): 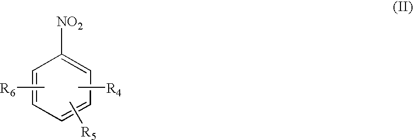
wherein:
R₄ is chosen from:
an amino radical optionally substituted with one or two groups chosen from (C₁-C₄)alkyl groups, (C₁-C₄)monohydroxyalkyl groups, (C₂-C₄)polyhydroxyalkyl groups, (C₁-C₄)aminoalkyl groups, mono((C₁-C₄)alkyl)amino((C₁-C₄)alkyl) groups, di((C₁-C₄)alkyl)amino((C₁-C₄)alkyl) groups, (C₁-C₄)ureidoalkyl groups a hydroxyl group and aryl groups; and
aryl groups optionally substituted with at least one group chosen from a hydroxyl group, a carboxyl group, an amino group and di((C₁-C₄)alkyl)amino groups;
R₅ is chosen from:
a hydrogen atom; a hydroxyl group; (C₁-C₄)alkyl groups; C₁-C₄ alkoxy groups; (C₁-C₄)monohydroxyalkyl groups; (C₂-C₄)polyhydroxyalkyl groups; (C₁-C₄)monohydroxyalkoxy groups; (C₂-C₄)polyhydroxyalkoxy groups; (C₁-C₄)aminoalkoxy groups; an amino radical optionally substituted with one or two groups chosen from (C₁-C₄)alkyl groups, (C₁-C₄)monohydroxyalkyl groups, (C₂-C₄)polyhydroxyalkyl groups, (C₁-C₄)aminoalkyl groups, mono((C₁-C₄)alkyl)amino((C₁-C₄)alkyl) groups, di((C₁-C₄)alkyl)amino((C₁-C₄)alkyl) groups, (C₁-C₄)ureidoalkyl groups and aryl groups; and aryl groups optionally substituted with at least one group chosen from a hydroxyl group, a carboxyl group, an amino group and di((C₁-C₄)alkyl)amino groups;
R₆ is chosen from: a hydrogen atom; a halogen atom; (C₁-C₄)alkyl groups; and a nitro group.

10. A composition according to claim 1, wherein said nitrobenzene direct dyes are chosen from:
2-amino-4-methyl-5-N-(β-hydroxyethyl)aminonitrobenzene,
4-N-(β-ureidoethyl)aminonitrobenzene,
4-(N-ethyl-N-β-hydroxyethyl)amino-1-N-(β-hydroxyethyl)aminonitrobenzene,
2-N-(β-hydroxyethyl)amino-5-methylnitrobenzene,
5-chloro-3-N-(ethyl)amino-4-hydroxynitrobenzene,
5-amino-3-chloro-4-hydroxynitrobenzene,
2-N-(γ-hydroxypropyl)amino-5-N,N-bis(β-hydroxyethyl)aminonitrobenzene,
5-hydroxy-2-N-(γ-hydroxypropyl)aminonitrobenzene,
1,3-bis(β-hydroxyethyl)amino-4-chloro-6-nitrobenzene,
2,4-diaminonitrobenzene,
3,4-diaminonitrobenzene,
2,5-diaminonitrobenzene,
3-amino-4-hydroxynitrobenzene,
4-amino-3-hydroxynitrobenzene,
5-amino-2-hydroxynitrobenzene,
2-amino-5-hydroxynitrobenzene,
4-amino-3-hydroxynitrobenzene,
5-amino-2-hydroxynitrobenzene,
2-amino-3-hydroxynitrobenzene,
2-amino-5-N-(β-hydroxyethyl)aminonitrobenzene,
2-amino-5-N,N-bis(β-hydroxyethyl)aminonitrobenzene,
2,5-N,N′-bis(β-hydroxyethyl)aminonitrobenzene,
2-N-(β-hydroxyethyl)amino-5-N,N-bis(β-hydroxyethyl)aminonitrobenzene,
2-amino-5-N-(methyl)aminonitrobenzene,
2-N-(methyl)amino-5-N,N-bis(β-hydroxyethyl)aminonitrobenzene,
2-N-(methyl)amino-5-(N-methyl-N-β-hydroxyethyl)aminonitrobenzene,
2,5-N,N′-(β-hydroxyethyl)aminonitrobenzene,
2-N-(β-hydroxyethyl)amino-5-hydroxynitrobenzene,
3-methoxy-4-N-(β-hydroxyethyl)aminonitrobenzene,
2-N-(methyl)amino-4-β-hydroxyethyloxynitrobenzene,
2-amino-3-methylnitrobenzene,
2-N-(β-hydroxyethyl)amino-5-aminonitrobenzene,
2-amino-4-chloro-5-N-(β-hydroxyethyl)aminonitrobenzene,
2-amino-4-methyl-5-N-(β-hydroxyethyl)aminonitrobenzene,
2-amino-4-methyl-5-N-(methyl)aminonitrobenzene,
2-N-(β-hydroxyethyl)amino-5-methoxynitrobenzene,
2-amino-5-β-hydroxyethyloxynitrobenzene,
2-N-(β-hydroxyethyl)aminonitrobenzene,
3-amino-4-N-(β-hydroxyethyl)aminonitrobenzene,
3-β-hydroxyethyloxy-4-N-(β-hydroxyethyl)aminonitrobenzene,
2-N-(methyl)amino-4-β,γ-dihydroxypropyloxynitrobenzene,
2-N-(β-hydroxyethyl)amino-5-β-hydroxyethyloxynitrobenzene,
2-N-(β-hydroxyethyl)amino-5-β,γ-dihydroxypropyloxynitrobenzene,
2-hydroxy-4-N-(β-hydroxyethyl)aminonitrobenzene,
2-N-(methyl)amino-4-methyl-5-aminonitrobenzene,
2-amino-4-isopropyl-5-N-(methyl)aminonitrobenzene,
2-N-(methyl)amino-5-(N-methyl-N-β,γ-dihydroxypropyl)aminonitrobenzene,
3-N-(β-hydroxyethyl)amino-4-N-(β-hydroxyethyl)aminonitrobenzene,
2-amino-4-methyl-5-N-(β,γ-dihydroxypropyl)aminonitrobenzene,
2-amino-4-methyl-5-hydroxynitrobenzene,
2-N-(β-hydroxyethyl)amino-4-N-(β-hydroxyethyl)aminonitrobenzene,
2-amino-5-N-(β-aminoethyl)aminonitrobenzene,
2-N-(β-aminoethyl)amino-5-methoxynitrobenzene,
2-N-(methyl)amino-5-N-(β-aminoethyl)aminonitrobenzene,
2-N-(β-aminoethyl)amino-4-N,N-(dimethyl)aminonitrobenzene,
3-amino-4-N-(β-aminoethyl)aminonitrobenzene,
2-amino-4-methyl-5-N-(β-aminoethyl)aminonitrobenzene,
2-N-(β-aminoethyl)amino-5-N,N-bis(β-hydroxyethyl)aminonitrobenzene,
3-β-aminoethyloxy-4-aminonitrobenzene,
2-N-(methyl)amino-5-(N-δ-amino-n-butyl)aminonitrobenzene,
2-N-(γ-amino-n-propyl)amino-5-N,N-(dimethyl)aminonitrobenzene,
3-methoxy-4-N-(β-aminoethyl)aminonitrobenzene,
2-N-(β-aminoethyl)amino-5-aminonitrobenzene,
2-amino-4-chloro-5-N-(β-aminoethyl)aminonitrobenzene,
2-N-(β-aminoethyl)amino-4-methoxynitrobenzene,
2-N-(β-aminoethyl)aminonitrobenzene,
2-N-(β-aminoethyl)amino-5-N-(β-aminoethyl)aminonitrobenzene,
2-N-(β-aminoethyl)amino-4-β-hydroxyethyloxynitrobenzene,
3-β-hydroxyethyloxy-4-N-(β-aminoethyl)aminonitrobenzene,
2-amino-5-aminoethyloxynitrobenzene,
3-hydroxy-4-N-(β-aminoethyl)aminonitrobenzene,
2-N-(β-aminoethyl)amino-5-β-hydroxyethyloxynitrobenzene,
2-N-(β-aminoethyl)amino-4-hydroxynitrobenzene,
[2-N-hydroxy-2-N-(β-hydroxyethyl)amino]-3-nitro-6benzyloxy-2-ethylamine, and
[2-N-hydroxy-2-N-(β-hydroxypropyl)amino]-3-nitro-6-benzyloxy-2-ethylamine.

11. A composition according to claim 1, said nitrobenzene dyes are chosen from:
2-amino-4-methyl-5-N-(β-hydroxyethyl)aminonitrobenzene,
4-N-(β-ureidoethyl)aminonitrobenzene,
4-(N-ethyl-N-β-hydroxyethyl)amino-1-N-(β-hydroxyethyl)aminonitrobenzene,
2-N-(β-hydroxyethyl)amino-5-methylnitrobenzene,
5-chloro-3-N-(ethyl)amino-4-hydroxynitrobenzene,
5-amino-3-chloro-4-hydroxynitrobenzene,
2-N-(γ-hydroxypropyl)amino-5-N,N-bis(β-hydroxyethyl)aminonitrobenzene,
5-hydroxy-2-N-(γ-hydroxypropyl)aminonitrobenzene,
1,3-bis(β-hydroxyethyl)amino-4-chloro-6-nitrobenzene,
3,4-diaminonitrobenzene,
2-amino-5-hydroxynitrobenzene,
2-amino-3-hydroxynitrobenzene,
2-amino-5-N-(β-hydroxyethyl)aminonitrobenzene,
2-amino-5-N,N-bis(β-hydroxyethyl)aminonitrobenzene,
2-N-(β-hydroxyethyl)amino-5-N,N-bis(β-hydroxyethyl)aminonitrobenzene,
2-N-(β-hydroxyethyl)amino-5-hydroxynitrobenzene,
2-N-(β-hydroxyethyl)amino-5-aminonitrobenzene,
2-N-(β-aminoethyl)amino-4-methoxynitrobenzene, and
2-N-(β-aminoethyl)amino-5-β-hydroxyethyloxynitrobenzene.

12. A composition according to claim 1, wherein said cationic dyes are chosen from compounds of formulae (III), (IV) and (V): 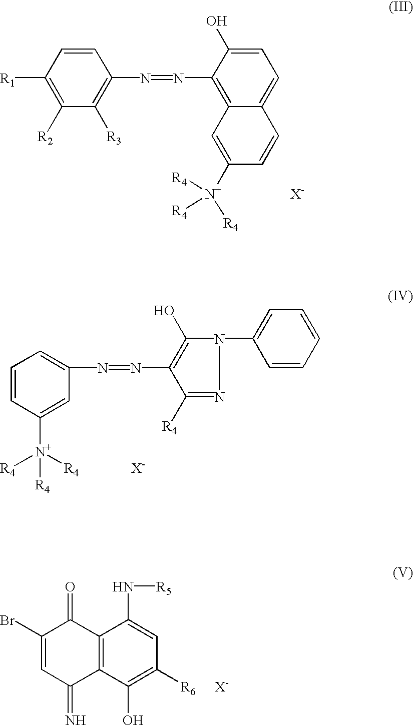
wherein:
R₁ is chosen from a hydrogen atom and an amino group;
R₂ is chosen from a hydrogen atom and a nitro group;
R₃ is chosen from a hydrogen atom, a nitro group and (C₁-C₄)alkoxy groups;
R₄ which are identical or different, are each chosen from (C₁-C₄)alkyl groups;
R₅ is chosen from a hydrogen atom and para-tri((C₁-C₄)alkyl)aminophenyl groups;
R₆ is chosen from a bromine atom and NH-para-tri((C₁-C₄)alkyl)aminophenyl groups; and
X⁻ is an anion; and mesomeric forms of the compounds of formulae (III), (IV) and (V).

13. A composition according to claim 12, wherein said X⁻ is an anion chosen from a chloride anion, a methylsulfate anion and an acetate anion.

14. A composition according to claim 1, wherein said cationic dyes are chosen:
8-[(4-aminophenyl)azo]-7-hydroxy-2-trimethylammonionaphthalene,
8-[(2-methoxyphenyl)azo]-7-hydroxy-2-trimethylammonionaphthalene,
8-[(4-amino-3-nitrophenyl)azo]-7-hydroxy-2-trimethylammonionaphthalene,
8-[(4-amino-2-nitrophenyl)azo]-7-hydroxy-2-trimethylammonionaphthalene,
3-[(3-methyl-5-hydroxy-1-phenyl-1H-pyrazol-4-yl)azo]trimethylammoniobenzene,
3-[(4-amino-6-bromo-5,8-dihydro-1-hydroxy-8-imino-5-oxo-2-naphthalenyl)amino]trimethylammoniobenzene, and
3-[(3,7-dibromo-5,8-dihydro-4-hydroxy-5-imino-8-oxo-1-naphthalenyl)amino]trimethylammoniobenzene.

15. A composition according to claim 1, wherein said cationic dyes are chosen from compounds of formulae(VI): 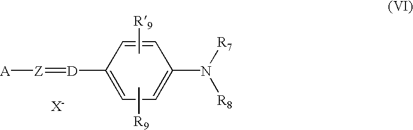
wherein:
Z and D, which are identical or different, are each chosen from a nitrogen atom and a —CH—group;
R₇ and R₈, which are identical or different are each chosen from a hydrogen atom, a 4′-aminophenyl group, and (C₁-C₄)alkyl groups, wherein said (C₁-C₄)alkyl groups are optionally substituted with a group chosen from a —CN group, a hydroxyl group and an —NH₂ group, and optionally wherein one of said R₇ and said R₈, together with a carbon atom of the benzene ring of formula (VI), forms a nitrogenous heterocycle optionally substituted with at least one group chosen from (C₁-C₄)alkyl groups, wherein said nitrogenous heterocycle optionally further comprises at least one heteroatom chosen from an oxygen atom and a nitrogen atom;
R₉ and R′₉, which are identical or different, are each chosen from a hydrogen atom, a halogen atom chosen from chlorine, bromine, iodine and fluorine, a cyano group, (C₁-C₄)alkyl groups, (C₁-C₄)alkoxy groups and acetyloxy groups;
X⁻ is an anion; and
A is chosen from groups of formulae A1 A2, A3, A4, A5, A6, A7, A8, A9, A10, A11, A12, A13, A14, A15, A16, A17, A18 and A19: 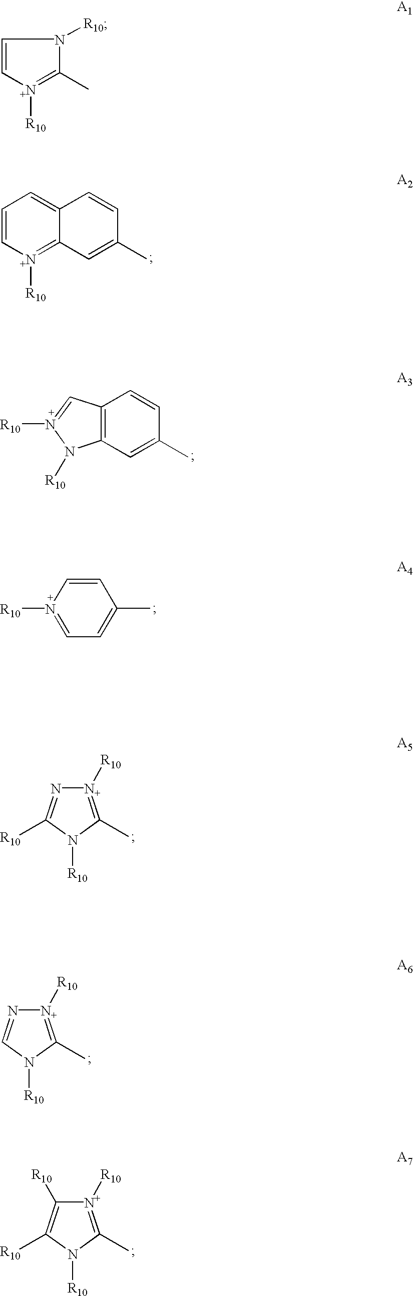
wherein:
R₁₀ is the same or different, and each is chosen from (C₁-C₄)alkyl groups optionally substituted with at least one hydroxyl radical; and
R₁₁ is chosen from (C₁-C₄)alkoxy groups.

16. A composition according to claim 1, wherein said cationic dyes are chosen from compounds of formulae(VII): 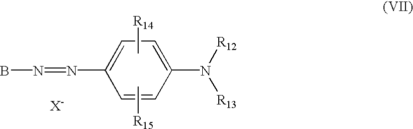
wherein:
R₁₂ is chosen from a hydrogen atom, and (C₁-C₄)alkyl groups;
R₁₃ is chosen from a hydrogen atom, a 4′-aminophenyl group, and (C₁-C₄)alkyl groups, wherein said (C₁-C₄)alkyl groups are optionally substituted with a group chosen from a —CN group and an amino group, and optionally wherein said R₁₃, together with said R₁₂, forms a nitrogenous heterocycle optionally substituted with at least one group chosen from (C₁-C₄)alkyl groups, wherein said nitrogenous heterocycle optionally further comprises at least one heteroatom chosen from an oxygen atom and a nitrogen atom;
R₁₄ and R₁₅, which are identical or different, are each chosen from a hydrogen atom, a halogen atom chosen from bromine, chlorine, iodine and fluorine, (C₁-C₄)alkyl groups, (C₁-C₄)alkoxy groups, and a —CN group;
X⁻ is an anion;
B is chosen from groups of formulae B1, B2, B3, B4, B5 and B6: 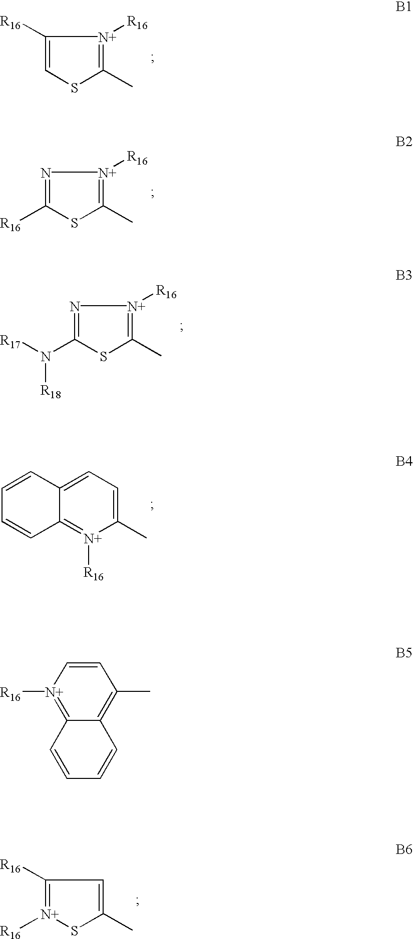
wherein:
R₁₆ is identical or different and each is chosen from (C₁-C₄)alkyl groups, and
R₁₇ and R₁₈, which are identical or different, are each chosen from a hydrogen atom and (C₁-C₄)alkyl groups.

17. A composition according to claim 1, wherein said cationic dyes are chosen from compounds of formulae(VIII) and (VIII′): 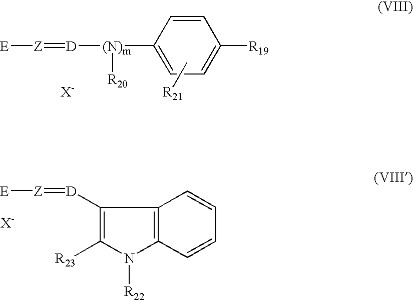
wherein:
Z and D, which are identical or different, are each chosen from a nitrogen atom and a —CH—group;
R₁₉ is chosen from a hydrogen atom, (C₁-C₄)alkoxy groups, an amino group, and a halogen atom chosen from bromine, chlorine, iodine and fluorine;
R₂₀ is chosen from a hydrogen atom, and (C₁-C₄)alkyl groups and optionally, wherein said R₂₀, together with a carbon atom of the benzene ring forms a nitrogenous heterocycle, wherein said nitrogenous heterocycle is optionally substituted with at least one group chosen from (C₁-C₄)alkyl groups, and optionally, wherein said nitrogenous heterocycle further comprises a member, wherein said member is an oxygen atom;
R₂₁ is chosen from a hydrogen atom and a halogen atom chosen from bromine, chlorine, iodine and fluorine;
R₂₂ and R₂₃, which are identical or different, are each chosen from a hydrogen atom and (C₁-C₄)alkyl groups;
m is an integer equal to 0 or 1;
X⁻ is an anion;
E is chosen from groups of formulae E1, E2, E3, E4, E5, E6, E7, and E8: 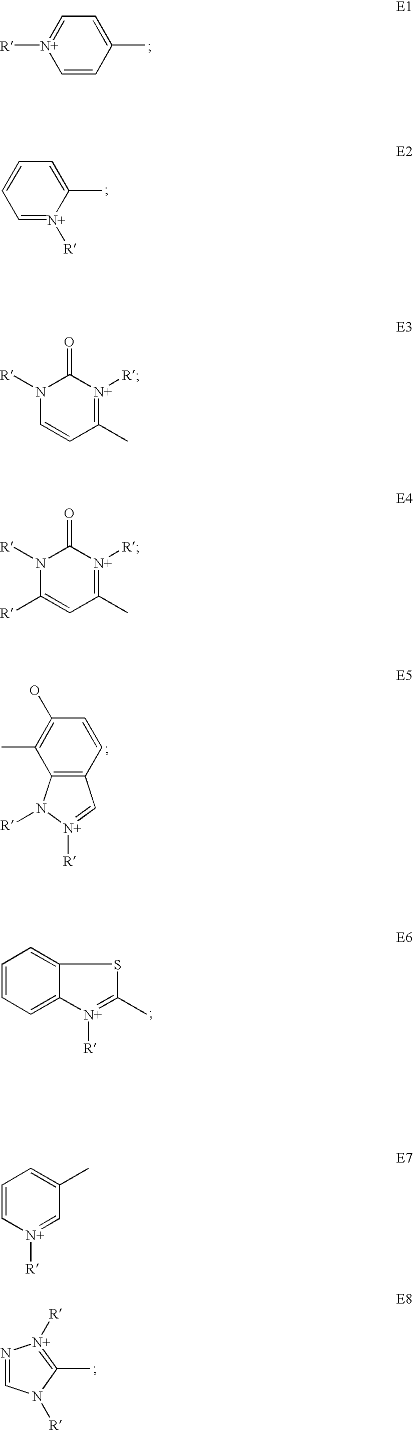
wherein:
R′ is identical or different, and each is chosen from (C₁-C₄)alkyl groups; and provided that:
when m is equal to 0 and D is a nitrogen atom, then E is a group of formula E9: 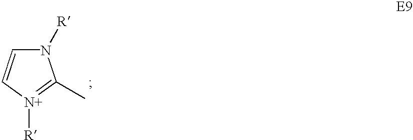
wherein:
R′, which are identical or different, are each chosen from (C₁-C₄)alkyl groups;

18. A composition according to claim 1, wherein said cationic dyes are chosen from compounds of formulae(IX):
G—N═N—J (IX)
wherein:
G is chosen from groups of formulae G₁, G₂ and G₃: 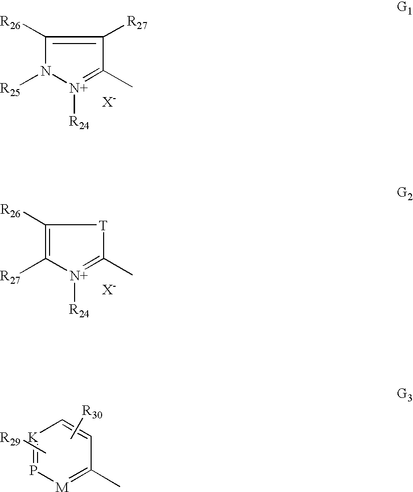
wherein:
R₂₄ is chosen from (C₁-C₄)alkyl groups, a phenyl group, wherein said phenyl group is optionally substituted with a group chosen from (C₁-C₄)alkyl groups, and a halogen atom chosen from chlorine, bromine, iodine and fluorine;
R₂₅ is chosen from (C₁-C₄)alkyl groups and a phenyl group;
R₂₆ is chosen from (C₁-C₄)alkyl groups, a hydrogen atom and a phenyl group;
R₂₇ is chosen from (C₁-C₄)alkyl groups and a phenyl group; provided that:
when said R₂₆ is other than a hydrogen atom, R₂₆ and R₂₇ optionally form a benzene ring, wherein said benzene ring is optionally substituted with at least one group chosen from (C₁-C₄)alkyl groups, (C₁-C₄)alkoxy groups, and a NO₂ group;
T is chosen from an oxygen atom, a sulfur atom and a group —NR₂₅, wherein R₂₅ is defined as above;
M is chosen from a —CH group, a —CR group, wherein R is chosen from (C₁-C₄)alkyl groups, and —N⁺R₂₈(X⁻)ᵣ groups, wherein R₂₈ is chosen from an O⁻, (C₁-C₄)alkoxy groups, and (C₁-C₄)alkyl groups and r is an integer equal to 0 or 1;
K is chosen from a —CH group, a —CR group, wherein R is chosen from (C₁-C₄)alkyl groups, and —N⁺R₂₈(X⁻)ᵣ groups, wherein R₂₈ is chosen from an O⁻, (C₁-C₄)alkoxy groups, and C₁-C₄ alkyl groups and r is an integer equal to 0 or 1;
P is chosen from a —CH group, a —CR group, wherein R is chosen from (C₁-C₄)alkyl groups; and —N⁺R₂₈(X⁻)ᵣ groups, wherein R₂₈ is chosen from an O⁻, (C₁-C₄)alkoxy groups, and (C₁-C₄)alkyl groups and r is an integer equal to 0 or 1;
R₂₉ and R₃₀, which are identical or different, are each chosen from a hydrogen atom, a halogen atom chosen from chlorine, bromine, iodine and fluorine, (C₁-C₄)alkyl groups, (C₁-C₄)alkoxy groups and an —NO₂ group;
X⁻ is an anion; and
J is chosen from:
(a) a group of formula J₁: 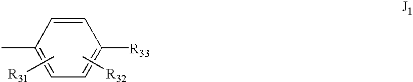
wherein:
R₃₁ is chosen from a hydrogen atom, a halogen atom chosen from chlorine, bromine, iodine and fluorine, (C₁-C₄)alkyl groups, (C₁-C₄)alkoxy groups, a hydroxyl group, an —NO₂ group, —NHR₃₄ groups, —NR₃₅R₃₆ groups and —NHCO(C₁-C₄)alkyl groups, wherein said R₃₄, said R₃₅, and said R₃₆ are defined below;
R₃₂ is chosen from a hydrogen atom, a halogen atom chosen from chlorine, bromine, iodine and fluorine, (C₁-C₄)alkyl groups, and (C₁-C₄)alkoxy groups;
R₃₃ is chosen from a hydrogen atom, a hydroxyl group, —NHR₃₄ groups, and —NR₃₅R₃₆ groups, wherein said R₃₄, said R₃₅, and said R₃₆ are defined below;
R₃₄ is chosen from a hydrogen atom, (C₁-C₄)alkyl groups, (C₁-C₄)monohydroxyalkyl groups, (C₂-C₄)polyhydroxyalkyl groups and a phenyl group;
R₃₅ and R₃₆, which are identical or different, are each chosen from (C₁-C₄)alkyl groups, (C₁-C₄)monohydroxyalkyl groups and (C₂-C₄)polyhydroxyalkyl groups;
wherein:
said R₃₁ and said R₃₂ optionally form a 5- or 6-membered ring, wherein said 5- or 6-membered ring optionally comprises at least one heteroatom chosen from a nitrogen atom, an oxygen atom, and a sulfur atom; and
said R₃₂ and one of said R₃₃ or said R₃₄ optionally form a 5- or 6-membered ring, wherein said 5- or 6-membered ring optionally comprises at least one heteroatom chosen from a nitrogen atom, an oxygen atom, and a sulfur atom; and
(b) a 5- or 6-membered nitrogenous heterocyclic group optionally comprising at least one unit chosen from heteroatoms and carbonyl-containing groups, wherein said 5- or 6-membered nitrogenous heterocyclic group is optionally substituted with at least one group chosen from C₁-C₄ alkyl groups, an amino group, and a phenyl group.

19. A composition according to claim 18, wherein said J chosen from said 5- or 6-membered nitrogenous heterocyclic group of (b) is chosen from a group of formula J₂: 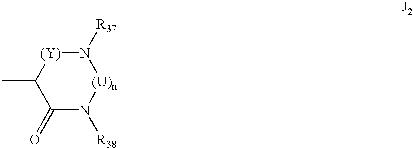
wherein:
R₃₇ and R₃₈, which are identical or different, are each chosen from a hydrogen atom, (C₁-C₄)alkyl groups and a phenyl group; 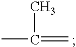
Y is chosen from a group —CO—and a group
n is an integer equal to 0 or 1; and provided that:
when n is equal to 1, then U is a —CO—group.

20. A composition according to claim 15, wherein said compounds of formula(VI) are chosen from the compounds of formula (VI₁) to (VI₅₄):

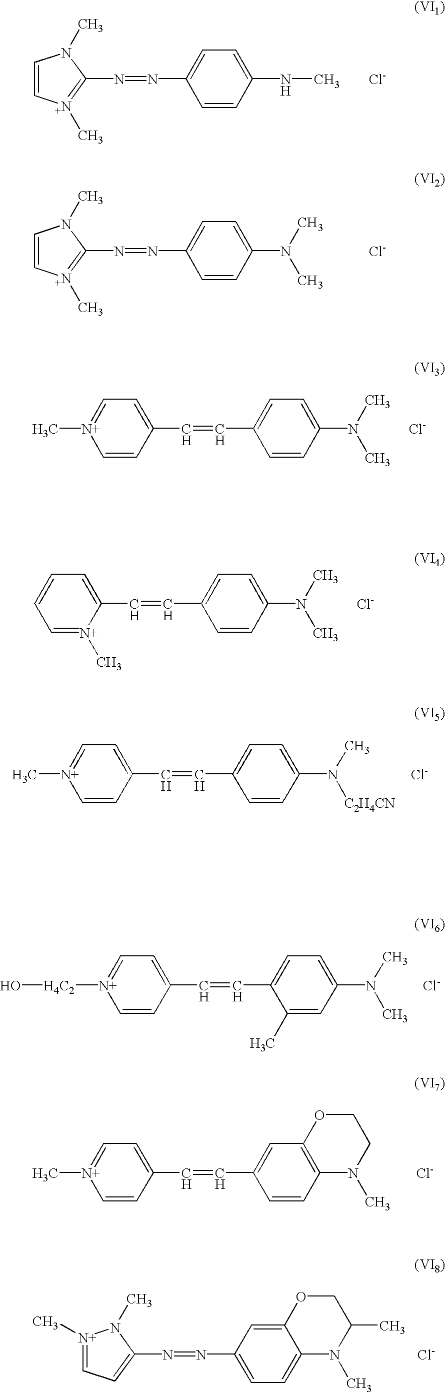

21. A composition according to claim 16, wherein said compounds of formula(VII) are chosen from the compounds of formula (VII₁) to (VII₉):

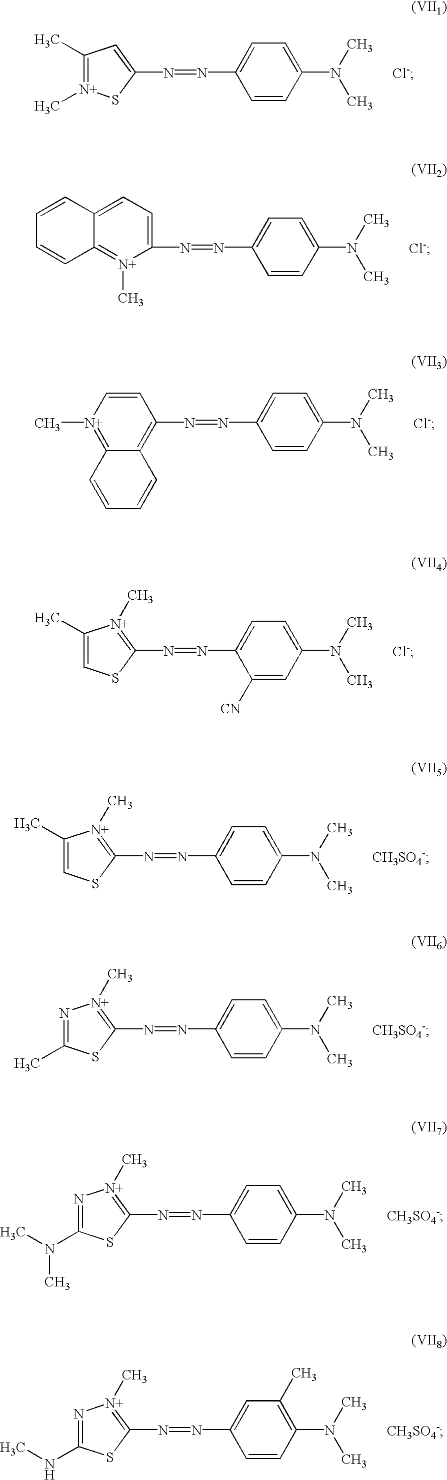

22. A composition according to claim 17, wherein said compounds of formula(VIII) are chosen from the compounds of formula (VIII₁) to (VIII₁₈):

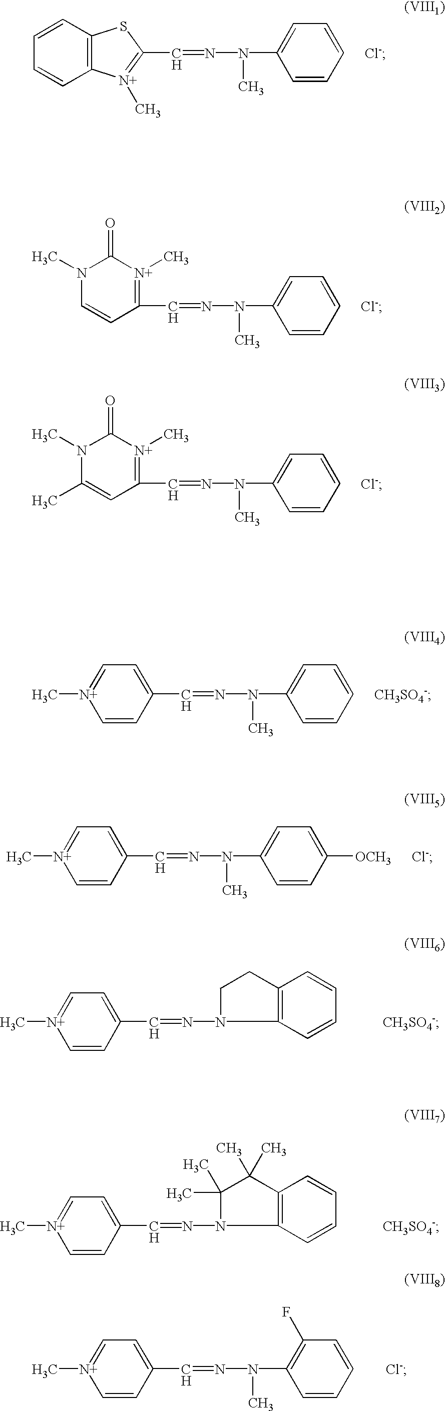

23. A composition according to claim 17, wherein said compounds of formula(VIII′) are chosen from the compounds of formula (VIII′₁) to (VIII′₃):

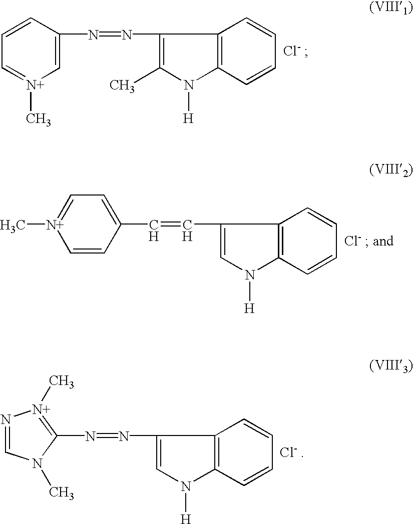

24. A composition according to claim 18, wherein said compounds of formula(IX) are chosen from the compounds of formula (IX₁) to (IX₇₇):

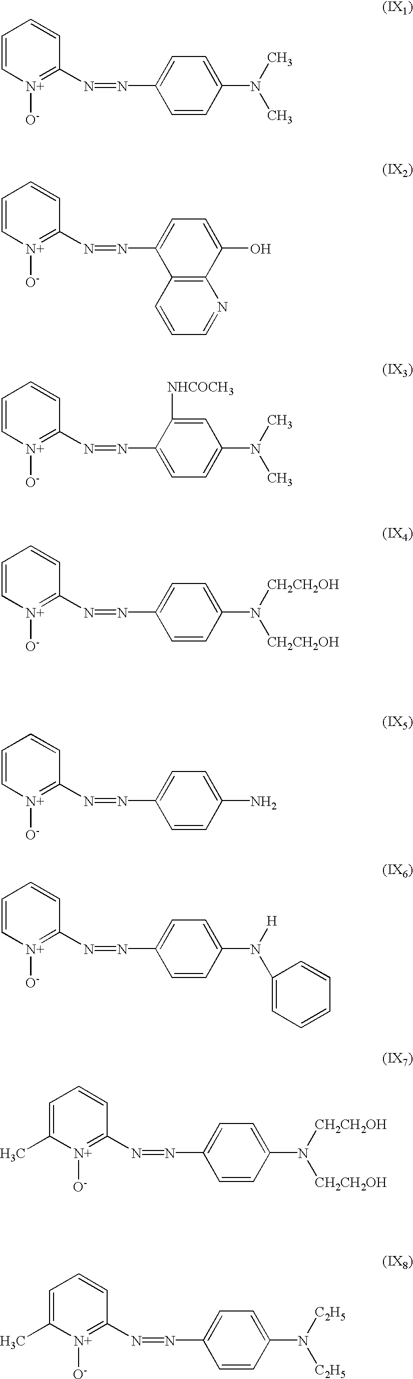

25. A composition according claim 1, wherein said at least one direct dye is present an amount ranging from 0.001 to 20% by weight relative to the total weight of the composition.

26. A composition according claim 25, wherein said at least one direct dye is present an amount ranging from 0.005 to 10% by weight relative to the total weight of the composition.

27. A composition according to claim 1 further comprising at least one coupler.

28. A composition according to claim 27, wherein said coupler is chosen from: meta-phenylenediamines, mneta-aminophenols, meta-diphenols, naphthols and heterocyclic couplers, such as, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and acid addition salts thereof.

29. A composition according to claim 27, wherein said coupler is chosen from: 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole,2,6-dimethylpyrazolo[1,5-b]-1,2,4-triazole, and acid addition salts thereof.

30. A composition according to claims 27, wherein said at least one coupler is present in an amount ranging from 0.0001 to 15% by weight relative to the total weight of the composition.

31. A composition according claim 1 further comprising at least one additional oxidation base which is other than said at least one oxidation dye precursor.

32. A composition according to claim 31, wherein said at least one additional oxidation base is chosen from: para-phenylenediamine, para-tolylenediamine, 2-hydroxyethyl-para-phenylenediamine, 1-N,N-bis(2-hydroxyethyl)-para-phenylenediamine, para-aminophenols, ortho-phenylene diamines, ortho-aminophenols, double bases, and heterocyclic bases.

33. A composition according to claim 31, wherein said additional oxidation bases is present in an amount ranging from 0.0001 to 15% by weight relative to the total weight of said composition.

34. A composition according to claim 1, wherein said medium suitable for dyeing comprises water.

35. A composition according to claim 34, wherein said medium suitable for dyeing further comprises at least one cosmetically acceptable organic solvent.

36. A composition according to claim 35, wherein said at least one cosmetically acceptable organic solvent is chosen from alcohols, glycols and ethers of glycols.

37. A composition according to claim 35, wherein said at least one cosmetically acceptable organic solvent is present in an amount ranging from 1 to 40% by weight relative to the total weight of the composition.

38. A composition according to claim 1, further comprising sequestrants, UV-screening agents, waxes, cyclic and linear, branched and unbranched, optionally organomodified volatile and nonvolatile silicones, preservatives, ceramides, pseudoceramides, vegetable oils, mineral oils and synthetic oils, vitamins, provitamins, opacifiers, thickening agents, and cationic polymers.

39. A composition according to claim 1, further comprising at least one agent chosen from reducing agents and antioxidants.

40. A composition according to claim 39, wherein said reducing agents and antioxidants are chosen from sodium sulfite, thioglycolic acid, thiolactic acid, sodium bisulfite, dehydroascorbic acid, hydroquinone, 2-methylhydroquinone, tert-butylhydroquinone and homogentisic acid.

41. A composition according to claim 38, wherein said reducing agents and antioxidants are present in an amount ranging, from 0.05 to 1.5% by weight relative to the total weight of said composition.

42. A composition according to claim 1, further comprising at least one fatty alcohol.

43. A composition according to claim 42, wherein said at least one fatty alcohol is chosen from: cetyl alcohol, stearyl alcohol, and oleyl alcohol.

44. A composition according to claim 42, wherein said at least one fatty alcohol is present in an amount ranging from 0.001 to 20% by weight relative to the total weight of the composition.

45. A composition according to claim 1, further comprising at least one surfactant chosen from nonionic, anionic, cationic and amphoteric surfactants.

46. A composition according to claim 45, wherein said at least one surfactant is chosen from, nonionic surfactants.

47. A composition according to claim 45, wherein said at least one surfactant is present in an amount ranging from 0.1 to 20% by weight relative to the total weight of the composition.

48. A composition according to claim 1, further comprising at least one cationic polymer.

49. A composition according to claim 48, wherein said at least one cationic polymer is present in an amount ranging from 0.01% to 10% by weight relative to the total weight of the final composition.

50. A composition according to claim 49, wherein said at least one cationic polymer is present in an amount ranging from 0.05% to 5% by weight relative to the total weight of the final composition.

51. A composition according to claim 50, wherein said at least one cationic polymer is present in an amount ranging from 0.1% to 3% by weight relative to the total weight of the final composition.

52. A composition according to claim 1, wherein said composition is a liquid, a powder, a cream, a gel, or in any form suitable for dyeing keratinous fibers, wherein further said composition is optionally pressurized.

53. A ready-to-use composition for oxidation dyeing keratinous fibers comprising, in an medium suitable for dyeing:
(i) at least one oxidation dye precursor chosen from the 1-(4-aminophenyl)-pyrrolidines of formula (I) and acid addition salts thereof: 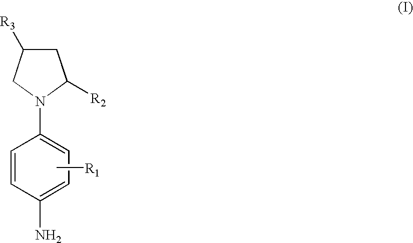
wherein:
R₁ is chosen from a hydrogen atom, ((C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and (C₂-C₅)polyhydroxyalkyl groups;
R₃ is chosen from a hydrogen atom, and a hydroxyl group; and
(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and
(iii) at least one oxidizing agent.

54. A ready-to-use composition according to claim 53, wherein the pH ranges from 3 to 12.

55. A ready-to-use composition according to claim 54, wherein the pH ranges from 6 to 11.

56. A ready-to-use composition according to claim 55, wherein said composition is a liquid, a powder, a cream, a gel, or in any form suitable for dyeing keratinous fibers, wherein further said composition is optionally pressurized.

57. A ready-to-use composition according to claim 53, wherein said at least one oxidizing agent is chosen from hydrogen peroxide, urea peroxide, alkali metal bromates, alkali metal ferricyanides, persalts, and oxidation-reduction enzymes optionally with their corresponding donors or cofactors if appropriate.

58. A ready-to-use composition according to claim 57, wherein said persalts are chosen from perborates and persulfates.

59. A ready-to-use composition according to claim 57, wherein said oxidation-reduction enzymes are chosen from laccases, peroxidases and 2 electron oxidoreductases.

60. A ready-to-use composition according to claim 57, wherein said at least one oxidizing agent further comprises a solution of hydrogen peroxide with a titre ranging from 1 to 40 in volume.

61. A ready-to-use composition according to claim 60, wherein said solution of hydrogen peroxide has a titre ranging from 5 to 40 in volume.

62. A method for oxidation dyeing keratinous fibers comprising:
(1) applying to said fibers at least one composition (A) for oxidation dyeing of keratinous fibers comprising, in a medium suitable for dyeing:
(i) at least one oxidation dye precursor chosen from 1-(4-aminophenyl)pyrrolidines of formula (I) and acid addition salts thereof: 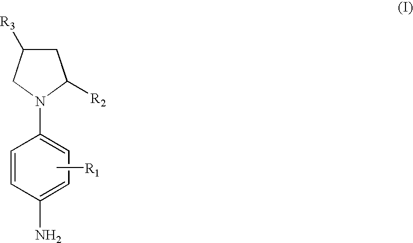
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and(C₂-C₅)polyhydroxyalkyl groups; and
R₃ is chosen from a hydrogen atom, and a hydroxyl group; and
(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and
(2) developing a color by applying to said fibers a composition (B) comprising at least one oxidizing agent, wherein:
said at least one composition (B) is combined at the time of use with said at least one composition (A) or said at least one composition (B) is applied simultaneously with or immediately after, applying said at lest one composition (A) without intermediate rinsing to said fibers.

63. A method for oxidation dyeing keratinous fibers, wherein said color is developed at alkaline, neutral or acidic pH.

64. A method for oxidation dyeing keratinous fibers comprising:
(1) applying to said fibers at least one composition (A) for oxidation dyeing of keratinous fibers comprising, in a medium suitable for dyeing:
(i) at least one oxidation dye precursor chosen from 1-(4-aminophenyl)pyrrolidines of formula (I) and acid addition salts thereof: 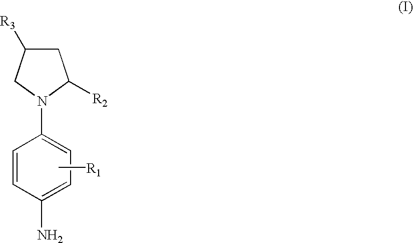
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and (C₂-C₅)polyhydroxyalkyl groups; and
R₃ is chosen from a hydrogen atom, and a hydroxyl group; and
(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and
(2) developing a color by applying to said fibers at least one composition (B) comprising at least one oxidizing agent, wherein said at least one composition (B) is combined at the time of use with said at least one composition (A) to form a combination;
(3) leaving said combination on said fibers for a time ranging from 1 to 60 minutes;
(4) rinsing said fibers and optionally shampooing and optionally further rinsing said fibers; and
(5) drying said fibers.

65. A method for oxidation dyeing keratinous fibers according to claim 64, wherein said time ranges from 10 to 40 minutes.

66. A kit comprising at least two compartments, wherein:
(1) a first compartment comprises:
(i) at least one oxidation dye precursor chosen from 1-(4-aminophenyl)pyrrolidines of formula (I) and acid addition salts thereof: 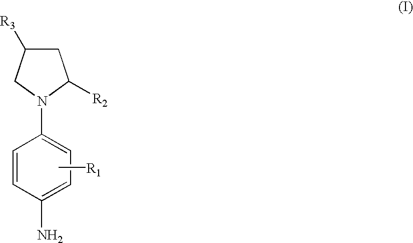
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and (C₂-C₅)polyhydroxyalkyl groups; and
R₃ is chosen from a hydrogen atom, and a hydroxyl group; and
(ii) at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and
(2) a second compartment comprising at least one oxidizing agent.

67. A kit comprising at least three compartments, wherein:
(1) a first compartment comprises at least one oxidation dye precursor chosen from 1-(4-aminophenyl)pyrrolidines of formula (I) and acid addition salts thereof: 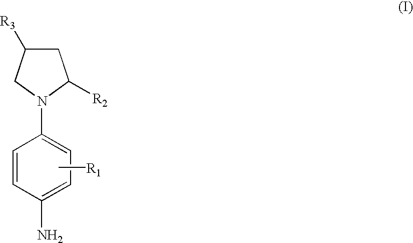
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and (C₂-C₅)polyhydroxyalkyl groups; and
R₃ is chosen from a hydrogen atom, and a hydroxyl group;
(2) a second compartment comprises at least one direct dye chosen from nitrobenzene dyes and cationic dyes, wherein said cationic dyes comprise a quaternized nitrogen atom and a —Z═D— group wherein, Z and D, which are identical or different, are each chosen from a nitrogen atom and a —CH— group; and
(3) a third compartment comprises at least one oxidizing agent.

68. A composition according to claim 1, wherein said cationic dyes are chosen from compounds of formulae(IX):
G—N═N —J (IX)
wherein:
G is chosen from groups of formulae G₁, G₂ and G₃: 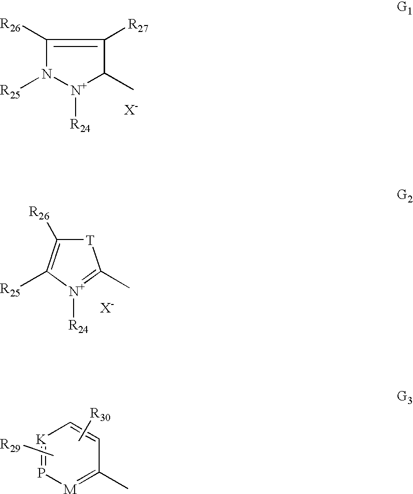
wherein:
R₂₄ is chosen from (C₁-C₄)alkyl groups, a phenyl group, wherein said phenyl group is optionally substituted with a group chosen from (C₁-C₄)alkyl groups, and a halogen atom chosen from chlorine, bromine, iodine and fluorine;
R₂₅ is chosen from (C₁-C₄)alkyl groups and a phenyl group;
R₂₆ is chosen from (C₁-C₄)alkyl groups, a hydrogen atom and a phenyl group;
R₂₇ is chosen from (C₁-C₄)alkyl groups and a phenyl group; provided that:
when said R₂₆ is other than a hydrogen atom, R₂₆ and R₂₇ optionally form a benzene ring, wherein said benzene ring is optionally substituted with at least one group chosen from (C₁-C₄)alkyl groups, (C₁-C₄)alkoxy groups, and a NO₂ group;
T is chosen from an oxygen atom, a sulfur atom and a group —NR₂₅, wherein R₂₅ is defined as above;
M is chosen from a —CH group, a —CR group, wherein R is chosen from (C₁-C₄)alkyl groups, and —N⁺R₂₈(X⁻)ᵣ groups, wherein R₂₈ is chosen from an O⁻, (C₁-C₄)alkoxy groups, and (C₁-C₄)alkyl groups and r is 0;
K is chosen from a —CH group, a —CR group, wherein R is chosen from (C₁-C₄)alkyl groups, and —N⁺R₂₈(X⁻)ᵣ groups, wherein R₂₈ is chosen from an O⁻, (C₁-C₄)alkoxy groups, and C₁-C₄ alkyl groups and r is 0;
P is chosen from a —CH group, a —CR group, wherein R is chosen from (C₁-C₄)alkyl groups; and —N⁺R₂₈(X⁻)ᵣ groups, wherein R₂₈ is chosen from an O⁻, (C₁-C₄)alkoxy groups, and (C₁-C₄)alkyl groups and r is 0;
R₂₉ and R₃₀, which are identical or different, are each chosen from a hydrogen atom, a halogen atom chosen from chlorine, bromine, iodine and fluorine, (C₁-C₄)alkyl groups, (C₁-C₄)alkoxy groups and an —NO₂ group;
X⁻ is an anion; and
J is chosen from:
(a) a group of formula J₁: 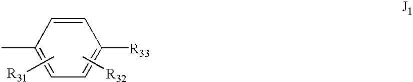
wherein:
R₃₁ is chosen from a hydrogen atom, a halogen atom chosen from chlorine, bromine, iodine and fluorine, (C₁-C₄)alkyl groups, (C₁-C₄)alkoxy groups, a hydroxyl group, an —NO₂ group, —NHR₃₄ groups, —NR₃₅R₃₆ groups and —NHCO(C₁-C₄)alkyl groups, wherein said R₃₄, said R₃₅, and said R₃₆ are defined below;
R₃₂ is chosen from a hydrogen atom, a halogen atom chosen from chlorine, bromine, iodine and fluorine, (C₁-C₄)alkyl groups, and (C₁-C₄)alkoxy groups;
R₃₃ is chosen from a hydrogen atom, a hydroxyl group, —NHR₃₄ groups, and —NR₃₅R₃₆ groups, wherein said R₃₄, said R₃₅, and said R₃₆ are defined below;
R₃₄ is chosen from a hydrogen atom, (C₁-C₄)alkyl groups, (C₁-C₄)monohydroxyalkyl groups, (C₂-C₄)polyhydroxyalkyl groups and a phenyl group;
R₃₅ and R₃₆, which are identical or different, are each chosen from (C₁-C₄)alkyl groups, (C₁-C₄)monohydroxyalkyl groups and (C₂-C₄)polyhydroxyalkyl groups;
wherein:
said R₃₁ and said R₃₂ optionally form a 5- or 6-membered ring, wherein said 5- or 6-membered ring optionally comprises at least one heteroatom chosen from a nitrogen atom, an oxygen atom, and a sulfur atom; and
said R₃₂ and one of said R₃₃ or said R₃₄ optionally form a 5- or 6-membered ring, wherein said 5- or 6-membered ring optionally comprises at least one heteroatom chosen from a nitrogen atom, an oxygen atom, and a sulfur atom; and
(b) a 5- or 6-membered nitrogenous heterocyclic group optionally comprising at least one unit chosen from heteroatoms and carbonyl-containing groups, wherein said 5- or 6-membered nitrogenous heterocyclic group is optionally substituted with at least one group chosen from C₁-C₄ alkyl groups, an amino group, and a phenyl group.
